(19) United States
(12) Patent Application Publication
(10) Pub. No.: US 2026/0256347 A1
(43) Pub. Date: Sep. 3, 2026

(54) FLUIDIC CLEANING EFFLUENT MANGEMENT ACCESSORY

(71) Applicant: Saban Ventures Pty Limited, Macquarie Park, NSW (AU)

(72) Inventors: Gilbert Tandang ALEMANA, Jordan Springs, NSW (AU); Rigor Del Mundo ASPA, Sutherland, NSW (AU); Danilo Pilapil BATERNA, Marsfield, NSW (AU); David Anthony PIDCOCK, Dulwich Hill, NSW (AU)

(21) Appl. No.: 19/165,840

(22) PCT Filed: Mar. 20, 2024
(86) PCT No.: PCT/IB2024/052684

§ 371 Date: Sep. 16, 2025

Related U.S. Application Data
(60) Provisional application No. 63/491,193, filed on Mar. 20, 2023.

(51) Int. Cl. A61B 1/12; A61B 90/70; A61L 2/18; A61L 2/20

(52) U.S. Cl. CPC A61B 1/123; A61B 90/70; A61L 2/18; A61L 2/20; A61B 2090/701

(57) ABSTRACT

Presented herein are techniques to manage a fluidic effluent being discharged in relation to a lumen cleaning process. More specifically, in accordance with certain embodiments presented, an effluent management accessory (EMA) is provided substantially separate gas portions from non-gas (e.g., liquid and/or solid) portions of the fluidic effluent discharged in association with, for example, a lumen cleaning process; the separated portions can each then be disposed with appropriately.

### BACKGROUND

### Field of the Invention

The present invention generally relates to an accessory for use in association with the cleaning of interior lumens.

### Related Art

There are several different types of systems/devices that include interior conduits/lumens that can need cleaning. The lumens can include, for example, dental lines, food/drink lines, medical lumens, etc.

A number of different medical devices (medical instruments), in particular, can include interior lumens that can be used to perform diagnostic and/or surgical procedures. For example, an endoscope is a medical device that includes interior lumens that can be used to visually inspect hollow organs or body cavities, deliver/extract fluids, etc. Specially designed endoscopes are used for different examinations, such as bronchoscopy, cystoscopy, gastroscopy, and proctoscopy. Endoscopes, as well as other available diagnostic and/or surgical medical devices are re-useable across multiple patients and, as such, the interior lumens must be cleaned between uses.

### SUMMARY

In one aspect, an apparatus is provided. The apparatus comprises: at least one input port configured to receive a fluidic effluent from at least one fluidic effluent source; a first stage separator configured to at least partially separate gas portions of the fluidic effluent from non-gas portions of the fluidic effluent, wherein the first stage separator produces a gaseous discharge and a non-gaseous discharge; and a second stage separator configured to receive the gaseous discharge and to separate gas portions of the gaseous discharge from non-gas portions of the gaseous discharge to produce a refined gaseous discharge.

In another aspect, an apparatus is provided. The apparatus comprises: a centrifugal separator comprising at least one input port configured to be connected to at a distal end of a lumen and to receive a fluidic effluent produced during cleaning of the lumen, wherein the centrifugal separator is configured to at least partially separate gas portions of the fluidic effluent from non-gas portions of the fluidic effluent, wherein the centrifugal separator produces a gaseous discharge and a non-gaseous discharge.

In another aspect, a method is provided. The method comprises: receiving, at a centrifugal separator, a fluidic effluent from a distal end of a lumen during a lumen cleaning process; and at the centrifugal separator, at least partially separating gas portions of the fluidic effluent from non-gas portions of the fluidic effluent to produce a gaseous discharge and a non-gaseous discharge.

### BRIEF DESCRIPTION OF THE DRAWING

Embodiments of the present invention are described herein in conjunction with the accompanying drawings, in which:

FIG. 1 is a schematic diagram illustrating an endoscope with interior lumens that can be cleaned in association with an effluent management accessory, in accordance with certain embodiments presented herein.

FIG. 2A is a flowchart of an example method for cleaning an interior lumen of a medical device using contaminant-detaching fluidic composition generating an effluent that can be managed with an effluent management accessory, in accordance with certain embodiments presented herein.

FIG. 2B is a schematic diagram illustrating a centrifugal separation stage/phase of a process for cleaning a lumen with a contaminant-detaching fluidic composition.

FIG. 2C is a schematic diagram illustrating a filtration/phase of a process for cleaning a lumen with a contaminant-detaching fluidic composition.

FIG. 3A is a schematic diagram illustrating use of an effluent management accessory with a lumen cleaning device, in accordance with certain embodiments presented herein;

FIG. 3B is a schematic diagram illustrating use of an effluent management accessory integrated in a lumen cleaning device, in accordance with certain embodiments presented herein;

FIGS. 4A, 4B, 4C, 4D, 4E, 4F, and 4G are diagrams illustrating an example effluent management accessory, in accordance with certain embodiments presented herein;

FIGS. 5A and 5B are top and bottom perspective views, respectively, illustrating the effluent management accessory of FIGS. 4A-4G mounted to a sink assembly, in accordance with certain embodiments presented herein;

FIGS. 6A, 6B, 6C, 6D, and 6E are diagrams illustrating another example effluent management accessory, in accordance with certain embodiments presented herein;

FIGS. 7A, 7B, and 7C are diagrams illustrating operation of a ball valve of the effluent management accessory of FIGS. 6A-6E, in accordance with certain embodiments presented herein;

FIGS. 8A, 8B, 8C, 8D, 8E, and 8F are diagrams illustrating another example effluent management accessory, in accordance with certain embodiments presented herein;

FIGS. 8G and 8H are a diagrams illustrating operation of a pinch valve of the effluent management accessory of FIGS. 8A-8F, in accordance with certain embodiments presented herein; and

FIG. 9 is a flowchart of an example method, in accordance with certain embodiments presented herein.

### DETAILED DESCRIPTION

As noted, there are a number of different types of systems/devices that include interior conduits/lumens, such as dental lines, food/drink lines, medical lumens, etc., that may need periodic and/or regular cleaning. For ease of description, the techniques presented herein are primarily described with reference to cleaning specific medical lumens, namely the interior lumens of an endoscope. However, it is to be appreciated that the techniques presented herein can also or alternatively be used to clean any type of interior lumen.

An endoscope is an elongate tubular medical device that may be rigid or flexible and which incorporates an optical or video system and light source. Typically, an endoscope is configured so that one end can be inserted into the body of a patient via a surgical incision or via one of the natural openings of the body. Internal structures near the inserted end of the endoscope can thus be viewed by an external observer.

As well as being used for investigation, endoscopes are also used to carry out diagnostic and surgical procedures. Endoscopic procedures are increasingly popular as they are minimally invasive in nature and provide a better patient outcome (through reduced healing time and exposure to infection) enabling hospitals and clinics to achieve higher patient turnover.

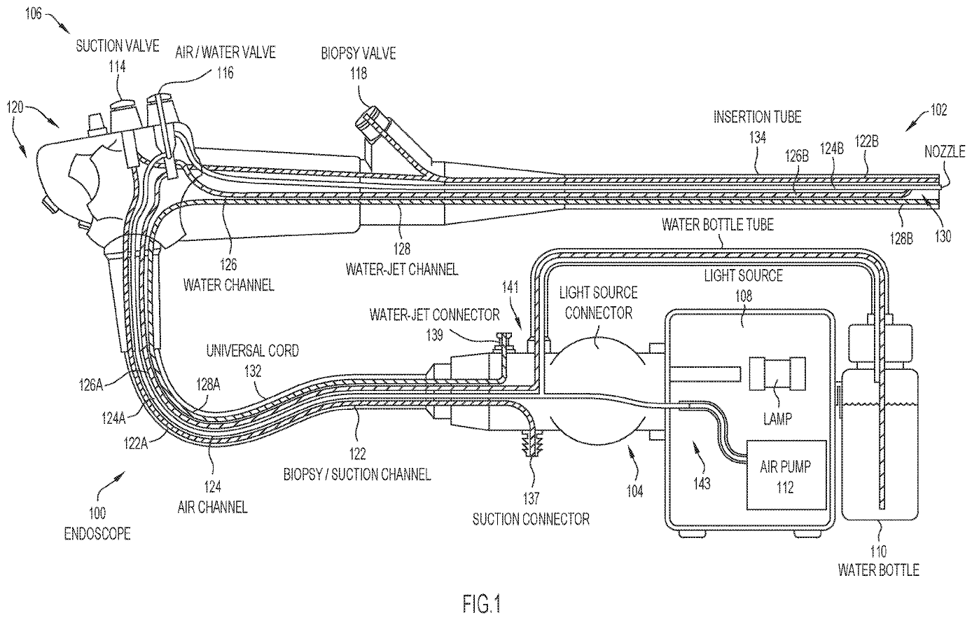

FIG. 1 is a schematic diagram of an example endoscope 100 with which aspects of the techniques presented herein can be implemented. As shown, endoscope 100, similar to most endoscopes, has a long tube-like structure with a distal end/tip 102 at one end for insertion into a patient and an opposing proximal or connector end 104, with a control handle 106 located between the two ends (e.g., generally at the center of the length between connector end 104 and distal end 102). The connector end 104 includes a plurality of connectors that enable the endoscope to be attached to, for example, a light source 108, water source 110, a suction source (not shown in FIG. 1), and a pressurized air source 112. For example, shown in FIG. 1, is a suction port/connector 137, a water-jet (auxiliary) port/connector 139, a water port/connector 141, and an air-port/connector 143. The control handle 106 is held by the operator during the procedure to control the endoscope 100 via valves, which include in this example a suction valve 114, an air/water valve 116, and a biopsy valve 118, and control wheels 120.

As shown in FIG. 1, endoscope 100 includes internal channels used either for delivering air and/or water, providing suction or allowing access for forceps and other medical equipment required during the procedure. As such, the distal end 102 contains the camera lens (not shown in FIG. 1), and the exits for the lighting, air, and water, as well as exits for suction and forceps. Some of the internal channels run from one end of the endoscope 100 to the other, while others run via valve sockets at the control handle. Some channels bifurcate while and others join from two into one.

More specifically, shown in FIG. 1 is a biopsy/suction channel 122, an air channel 124, a water channel 126, and a water-jet channel 128. The biopsy/suction channel 122 includes two sections, referred to as proximal section 122A and distal section 122B that are connected via the suction valve 114. The air channel 124 also includes two sections, referred to as proximal section 124A and distal section 124B that are connected via the air/water valve 116. Similarly, the water channel 126 also includes two sections, referred to as proximal section 126A and distal section 126B that are connected via the air/water valve 116. The distal section 126B of the water channel joins to the distal section 124B of the air channel at a location 130 within the distal end 102. The water-jet channel 128 extends directly from the connector end 104 to the distal end 102 (via the control handle 106) but is similarly referred to as having a proximal section 128A and distal section 128B. The proximal sections 122A, 124A, 126A, and 128A of the channels are sometimes referred to as being located within a universal cord section (cord) 132 of the endoscope 100, while the distal sections 122B, 124B, 126B, and 128B of the channels are sometimes referred to as being located within an insertion tube 134 of the endoscope. More generally, as used herein, the proximal sections 122A, 124A, 126A, and 128A are the portions of the channels located between the connector end 104 and a valve (e.g., valve 114 or 116) at the control handle 106 and/or a mid-point of the control handle 106, as applicable. The distal sections 122B, 124B, 126B, and 128B are the portions of the channels located between the valve (e.g., valve 114 or 116) at the control handle 106 and/or a mid-point of the control handle 106, and the distal end 102 of the endoscope 102.

The high cost of endoscopes means they must be re-used. As a result, because of the need to avoid cross infection from one patient to the next, each endoscope must be thoroughly cleaned and disinfected or sterilized after each use. This involves the cleaning of not only the outer of the endoscope 100, but also cleaning and disinfecting the internal channels/lumens (e.g., lumens 122, 124, 126, and 128 of FIG. 1).

Endoscopes used for colonoscopic procedures are typically between 2.5 and 4 meters long and have one or more lumen channels of diameter of no more than a few millimeters. Ensuring that such long narrow channels are properly cleaned and disinfected between patients presents a considerable challenge. The challenge of cleaning is also made more difficult by the fact that there is not just one configuration/type of endoscope. Indeed, there are a variety of endoscopic devices, each suited to a particular insertion application, such as colonoscopes inserted into the colon, bronchoscopes inserted into the airways, gastroscopes for investigation of the stomach, etc. Gastroscopes, for instance, are smaller in diameter than colonoscopes; bronchoscopes are smaller again and shorter in length while duodenoscopes have a different tip design to access the bile duct.

A variety of options are available to mechanically remove biological residues from the lumen which is the first stage in the cleaning and disinfection process. One procedure for cleaning the lumens utilizes small brushes mounted on long, thin, flexible lines. Brushing is the mandated means of cleaning the lumen in some countries. These brushes are fed into the lumens while the endoscope is submerged in warm water and a cleaning solution. The brushes are then pushed/pulled through the length of the lumens in an effort to scrub off the soil/bio burden. Manual back and forth scrubbing is typically required. Water and cleaning solutions are then flushed down the lumens. These flush-brush processes are repeated three times or until the endoscope reprocessing technician is satisfied that the lumen is clean. At the end of this cleaning process air is pumped down the lumens to dry them. A flexible pull-through device having wiping blades may also be used to physically remove material. A liquid flow through the lumen at limited pressure can also be used.

In general, however, only the larger suction/biopsy lumens (e.g., 122 in FIG. 1) can be cleaned by brushing or pull throughs. Air/water channels (e.g., channels 124 and 126) may be too small for brushes so these lumens are commonly only flushed with water and cleaning solution.

After mechanical cleaning, a chemical clean is carried out to remove the remaining biological contaminants. Because endoscopes are sensitive and expensive medical instruments, the biological residues cannot be treated at high temperatures or with strong chemicals. For this reason, the mechanical cleaning should be as thorough as possible. In many cases, the current mechanical cleaning methodologies fail to fully remove biofilm from lumens, particularly where cleaning relies on liquid flow alone. Regardless of how good the conventional cleaning processes are, it is not uncommon that a small microbial load will remain in the channel.

There is research showing that the method of cleaning with brushes, even when performed as prescribed, may not completely remove biofilm in endoscope lumens. As well as lacking in efficacy, the current manual brushing procedures can suffer from other drawbacks. The large number of different endoscope manufacturers and models results in many minor variations of the manual cleaning procedure. This can lead to confusion and ultimately poor compliance in cleaning processes. The current system of brushing can also be hazardous in that the chemicals that are currently used to clean endoscopes can adversely affect the reprocessing staff.

The current system of manual brushing can also be labor intensive, leading to increased cost. Thus, the current approaches to cleaning and disinfecting the lumens in medical cleaning apparatus are still inadequate and residual microorganisms are now recognized as a significant threat to patients and staff exposed to these devices. For example, there is evidence of bacterial transmission between patients from inadequate cleaning and disinfection of internal structures of endoscopes which in turn has led to patients acquiring mortal infections. Between 2010 and 2015 more than 41 hospitals worldwide, most in the U.S., reported bacterial infections linked to the scopes, affecting 300 to 350 patients (http://www.modernhealthcare.com/article/20167415/NEWS/167419935). It would be expected that a reduction in the bioburden in various medical devices would produce a concomitant overall reduction in infection rates and mortality.

In addition, if endoscopes are not properly cleaned and dried, biofilm can build up on the lumen wall. Biofilms start to form when a free-floating microorganism attaches itself to a surface and surrounds itself with a protective polysaccharide layer. The microorganism then multiplies, or begins to form aggregates with other microorganisms, increasing the extent of the polysaccharide layer. Multiple sites of attachment can in time join up, forming significant deposits of biofilm. Once bacteria or other microorganisms are incorporated in a biofilm, they become significantly more resistant to chemical and mechanical cleaning than they would be in their free-floating state. The organisms themselves are not inherently more resistant, rather, resistance is conferred by the polysaccharide film and the fact that microorganisms can be deeply embedded in the film and isolated from any chemical interaction. Any residual biofilm remaining after an attempt at cleaning quickly returns to an equilibrium state and further growth of microorganisms within the film continues. Endoscopes lumens are particularly prone to biofilm formation. They are exposed to significant amounts of bioburden, and subsequent cleaning of the long narrow lumens is quite difficult due to inaccessibility and the inability to monitor the cleaning process.

There is considerable pressure in medical facilities to reprocess endoscopes as quickly as possible. Because endoscopes are cleaned by hand, training and attitude of the technician are important in determining the cleanliness of the device. Residual biofilm on instruments can result in a patient acquiring an endoscope acquired infection. Typically, these infections occur as outbreaks and can have fatal consequences for patients.

The use of contaminant-detaching fluidic compositions propelled through respective lumens of a medical device has been found to be particularly effective at removing unwanted matter via physical contact-yet safely-interacting with the lumens to clean them. In these techniques, a liquid-powder mixture (contaminant-detaching fluidic composition) is created, apportioned into a suitable amount, and then delivered at a suitable velocity through at least a portion of the lumen. The liquid-powder mixture (e.g., contaminant-detaching fluidic composition) is sometimes referred to herein as a ‘slurry,’ and the apportioned amount of the liquid-powder mixture is sometimes referred herein to as a ‘cleaning slug’ or ‘slug.’

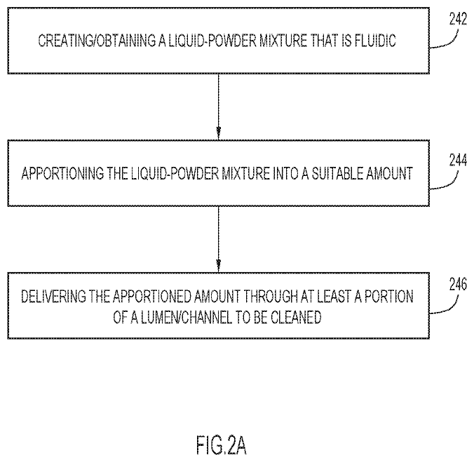

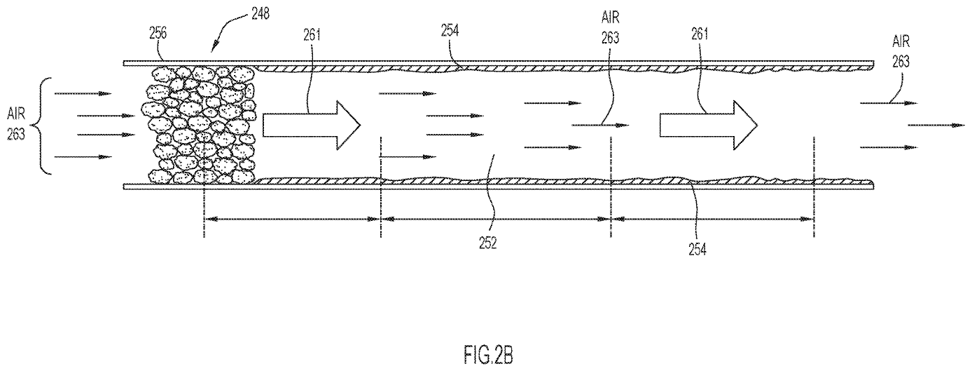

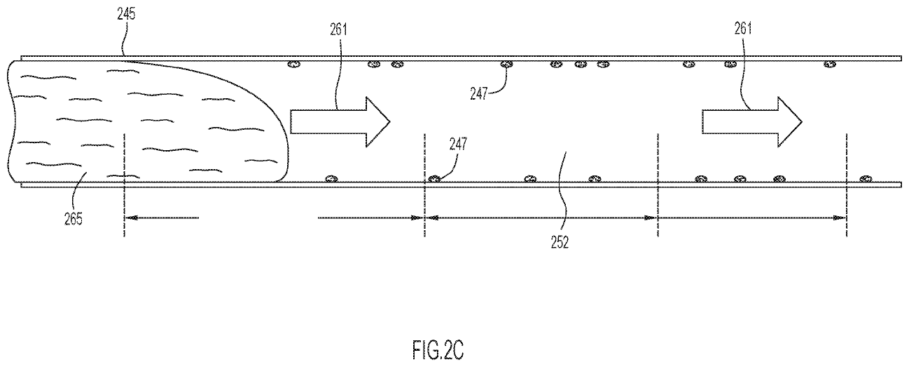

FIG. 2A illustrates an exemplary method 240 of cleaning a lumen of a medical device using a slurry and a cleaning slug. The method 240 of FIG. 2A begins at 242 with the creating, mixing, or otherwise obtaining of a liquid-powder mixture. At 244, the liquid-powder mixture is apportioned into a suitable amount. At 246, the apportioned amount of the liquid-powder mixture is delivered (e.g., propelled) through at least a portion of a lumen to be cleaned. This process may of course be implemented in any of a variety of ways.

For example, any suitable liquid-powder mixture may be implemented. As can be appreciated, the liquid component of the mixture can facilitate the fluidity of the mixture, while the presence of the powder can act to interact with (e.g., scour) the walls of the target lumen (e.g., channel) to thereby clean the lumen. In accordance with certain examples, the powder component of the liquid-powder mixture is present within the mixture in amounts greater than the respective saturation limit within the respective liquid, which can facilitate a cleaning interaction between the mixture and the walls of the lumen. In certain embodiments, the liquid-powder mixture comprises mixture of sodium bicarbonate powder and water, where the sodium-bicarbonate is present in an amount greater than the respective saturation level. For example, in a number of embodiments, sodium-bicarbonate can, at certain stages, be present in an amount greater than 10% of the mixture by mass. It has been determined that a mixture of sodium bicarbonate and water can be particularly effective in the disclosed application. Moreover, these constituent components are readily available. However, it would be appreciated that any suitable liquid-powder mixture can be used in alternative examples.

In some arrangements, the powder in the mixture is present in an amount below the respective saturation of the associated liquid. However, the liquid is delivered to the target lumen prior to the complete dissolution of the powder in the liquid. In this way, the undissolved powder can still interact with the target lumen to be cleaned.

Moreover, it is to be appreciated that the liquid-powder mixture can be created/obtained in any of a variety of ways. For example, in certain embodiments, a powder is obtained from a cartridge or other consumable chamber/container, water is obtained from a tap, and these constituent components are mixed within a holding chamber (or within the consumable chamber itself) proximate (e.g., within days or weeks) to the time of cleaning. This approach may be advantageous insofar as powders such as sodium bicarbonate can be relatively stable and can have a long shelf life and suitable sources of water are readily available. However, in other embodiments, the mixture may be obtained in an already mixed form.

As noted, method 240 involves apportioning the liquid-powder mixture into a suitable amount. As illustrated, the apportioned amounts are subsequently delivered through a lumen to be cleaned. Delivering discrete amounts of the mixture can be advantageous insofar as the discrete amounts can be delivered periodically at suitable velocities, and the periodic application of the composition can help facilitate the cleaning of the lumen while not clogging/blocking the target lumens. Moreover, the discrete nature of the delivered amounts can facilitate the maintenance of a suitable delivery velocity, which can also aid cleaning. For example, if the liquid-powder mixture was delivered continuously (and not in discrete, apportioned amounts), this approach might risk ‘clogging’ or otherwise obstructing the lumen to reduce the velocity at which the contaminant-detaching fluidic composition flows through the lumen, and can thereby impact cleaning efficacy.

Notably, different amounts of liquid-powder mixture may be differently suitable for the different characteristics of lumens to be cleaned. For example, air/water channels within an endoscope are typically amongst the narrowest lumens and, accordingly, may be more suitably cleaned with relatively smaller amounts of a liquid-powder mixture (whereas using larger amounts of a liquid-powder mixture may result in blocking such a narrow channel). In contrast, the suction/biopsy channels of an endoscope are typically amongst the widest lumens and, accordingly, may be more suitably cleaned with relatively larger amounts of liquid-powder mixture. As such, the amount of liquid-powder mixture apportioned for use in cleaning a given lumen is a function of the geometry of the lumen to be cleaned. It should of course be appreciated that the amount of liquid-powder mixture apportioned can also or alternatively be a function of any of a variety of parameters, including those that relate to the target.

The apportioned amount of the liquid-powder mixture can be determined in any of a variety of ways. For example, in certain embodiments, a valve may be used to draw a target amount of liquid-powder mixture from a reservoir. In some embodiments, a self-regulating pressurized system is used to draw a suitable amount of liquid-powder mixture from the reservoir.

As noted, method 240 of FIG. 2A further includes delivering the apportioned amount of liquid-powder mixture through at least a portion of the lumen to be cleaned. In general, a carrier fluid (e.g., air, water, etc.) is used to deliver (e.g., propel) the apportioned amount of the liquid-powder mixture through at least a portion of the lumen to be cleaned at a suitable velocity. The apportioned amount of the liquid-powder mixture is delivered in a manner (e.g., suitable size, suitable velocity, etc.) to provide an appropriate physical interaction between the mixture and the walls of the lumen, meaning that the undissolved powder will physically contact or run against the walls of the lumen to remove contaminants (e.g., bioburdens) therefrom. Of course, the apportioned amount of the liquid-powder mixture may be delivered through the lumen in any suitable way to enable cleaning of a lumen.

Notably, method 240 can be iterated any number of times to facilitate the cleaning of the lumen of a medical device. For example, FIG. 2B illustrates the delivery of one cleaning slug 248 (e.g., an apportioned amount of a liquid-powder mixture) through a lumen 252 to remove contaminants from the walls of the lumen, where the general direction of travel of the slug 248 is represented by arrows 261. That is, as shown, the lumen 252 has one or more contaminants 254 (e.g., bioburdens) disposed on the inner surface/walls 256 of the lumen. It is further illustrated that the cleaning slug 248 is delivered through the lumen 252 to physically interact with the walls of the lumen and thereby remove the contaminants 254 therefrom. The cleaning slug 248 can be considered to be entrained within a carrier fluid, which in this example comprises air (represented by arrows 263).

In general, cleaning slugs presented herein, such as cleaning slug 248, can have different forms/arrangements. For example, in certain embodiments, a cleaning slug presented herein can be a relatively singular/unitary mass (e.g., potentially substantially occluding the lumen while traveling therethrough), which is sometimes referred to herein as a “unitary slug.” However, in other embodiments, a cleaning slug can be an “agglomeration” or “cluster” of smaller masses/groups that travel through the lumen as a loose group (e.g., potentially not occluding the lumen while traveling therethrough), sometimes referred to herein as a “cluster slug.” FIG. 2B schematically illustrates an example in which the slugs 248 are cluster slugs.

In certain embodiments, a cleaning slug can transition between different forms during the slug's life cycle. For example, a slug could be apportioned (initially created) as a unitary slug, but then transition to a cluster slug. This transition could occur before entering the lumen (e.g., in a delivery chamber) and/or while traveling through the lumen.

As noted above, FIG. 2B generally illustrates the delivery of a cleaning slug 248 through a lumen 252. In certain examples, FIG. 2B represents a first stage/phase of a cleaning process, while FIG. 2C represents a second stage/phase of the cleaning process. More specifically, after a cleaning slug 248 is delivered through the lumen 252 (as in FIG. 2B), a fluid flow is delivered through the lumen 252 without any slugs. In the example of FIG. 2C, the fluid flow is comprised of water 265, where the general direction of travel is again represented by arrows 261. In certain examples, the fluid flow (e.g., water 265) is configured to remove residuals 247 from the lumen. The residuals 247 can comprise, for example, some remaining portion of the contaminant 254 and/or portions of the slugs 248 that may remain on the walls of the lumens 252 after passage of the slugs (e.g., the slug can break up into different clusters, some of which remain on the walls of the lumen 252). If present, portions of the slug 248 that may remain on the walls of the lumens 252 may aid in the cleaning process as these portions are flushed through the lumen 252 by the fluid flow.

FIGS. 2B and 2C generally illustrate an arrangement in which the second stage (fluid flow) is interspersed between the delivery of cleaning slugs. That is, in the embodiments of FIGS. 2B and 2C, the delivery of each cleaning slug is followed by a fluid-only flow. In certain alternative embodiments, multiple slugs could alternatively be delivered through a lumen either simultaneously or sequentially, without separation (e.g., without a fluid-only flow).

Importantly, while FIG. 2B illustrates that the two cleaning slugs 248 are simultaneously being delivered through the lumen 252, it should be appreciated that in certain embodiments, the cleaning slugs are delivered through the lumen sequentially (e.g., one-at-a-time). While FIG. 2B illustrates the delivery of two cleaning slugs 248, it should be appreciated that any number of cleaning slugs may be delivered through the lumen in different embodiments. In general, the use of a series of discrete/individual cleaning slugs 248, as opposed to a single large flow, can allow the individual cleaning slugs to maintain sufficient kinetic energy to pass through the lumens at a rate that allows the particles with the slugs to advantageously interact with, and remove contaminants from, the lumen walls.

As noted above, a lumen cleaning process, such as described above with reference to FIGS. 2A, 2B, and 2C, can be implemented in a number of different manners with a number of different lumens. For context, one specific example implementation is described with reference to cleaning at least part of the endoscope 100 of FIG. 1.

More specifically, in one example cleaning process/cycle, one (1) cleaning slug is fired/shot into the water-jet channel 128 via water-jet connector 138, nine (9) cleaning slugs are then fired into the biopsy/suction channel 122 via suction connector 137, one (1) cleaning slug is then fired into the water-jet channel 128 via water-jet connector 138, three (3) cleaning slugs are then fired into the distal section 122B of the biopsy/suction channel 122 via biopsy valve 118, one (1) cleaning slug is then fired into the water-jet channel 128 via water-jet connector 138, and then nine (9) cleaning slugs are fired into the biopsy/suction channel 122 via suction connector 137. The cleaning cycle can further include firing/shooting six (6) cleaning slugs into the air channel 124 via air connector 143 and firing six (6) cleaning slugs into the water channel 126 via water connector 141 (e.g., in parallel). The firing of the cleaning slugs within each target lumen can be followed by a fluid flow, as described above with reference to FIG. 2C. The cleaning slugs and fluid flows can be delivered via one, or possible multiple connectors (e.g., one connector for the air pipe and one connector for the air/water bottle).

In certain examples, approximately 180-200 grams of a slurry could be used to clean a typical flexible GI endoscope. For example, approximately use 80-100 grams can be used to clean a relatively large channel (e.g., suction/biopsy channel 122) with 21 shots in total and an approximately 15 second delay between each shot. For a relatively small channel (e.g., air/ water channels), the process can use approximately 60-80 grams with 12 shots in total and an approximately 30 second delay between each shot. For other small channels (e.g., water-jet channel 128), the process can use approximately 10-20 grams with 3 shots in total and an approximately 30 second delay between each shot. Again, each of these channels can also receive a subsequent fluid flow (e.g., after each cleaning slug), as described above with reference to FIG. 2C.

As noted above, cleaning slugs are delivered to a target lumen with a velocity that is suitable/sufficient to remove contaminants from the walls of the target lumen. The velocity of the cleaning slugs can vary, for example, based on the attributes of the target lumen, the attributes of the of the contaminant-detaching fluidic composition (slurry) used to form the slug, etc. In one illustrative example, the slug velocity for a relatively large lumen may be around 1000 mm/second.

In addition, the cleaning slugs can be delivered within specific pressure and fluid flow (air) ranges. In certain examples, the cleaning slugs can be delivered with a pressure up to approximately 26 psi (air, note this is regulated by a PPR as described below), up to approximately 24 psi (water), etc. Example air flow metrics can include approximately 50 SLPM (large channel no load), approximately 11-17 SLPM (large channel during dosing), approximately 7-10 SLPM (large channel during full load), approximately 5-7 SLPM (small channel no load), and approximately 0.1 SLPM (small channel during full load). It is to be appreciated that these ranges and values are merely illustrative.

As described elsewhere herein, there are various techniques to clean a lumen, including a manual process (e.g., manual scrubbing/brushing that is preceded and/or followed by a flushing fluid flow), a process using a propelled contaminant-detaching fluidic composition, a process that involves a water or other fluid flushing flow without brushing/scrubbing, and/or other techniques. However, all of these techniques result, at least in certain stages, in some discharge that is referred to herein as “fluidic effluent” that exits from, for example, a distal end of the lumen. The fluidic effluent can include the fluids (e.g., air, water, blood, contaminant-detaching fluidic composition, etc.) and/or solids (e.g., powder, biofilm, etc.) used in the cleaning process and/or the contaminants removed from the lumen during the cleaning process. That is, as used herein, the fluidic effluent can include water only, air only, the combination of water and air, water and/or air in combination with contaminants, and so on, regardless of the source, particular flushing medium/technique, etc.

Presented herein are techniques to manage a fluidic effluent (fluidic cleaning effluent) being discharged in relation to a lumen cleaning process. More specifically, in accordance with certain embodiments presented, an effluent management accessory (EMA) is provided to substantially separate gas portions from non-gas (e.g., liquid and/or solid) portions of the fluidic effluent discharged in association with, for example, a lumen cleaning process; the separated portions can each then be disposed with appropriately. The effluent management accessory can thereby promote the high quality of the environment for lab/cleaning personnel. The value of maintaining/promoting a high-quality environment for cleaning in this context has been hitherto underappreciated, and the disclosed apparatus provide an elegant solution for doing so.

As noted, merely for ease of illustration, the techniques presented herein are primarily described with reference to cleaning a specific type of medical lumen, namely the channels of an endoscope, via an automated cleaning process using a fluidic composition and a lumen cleaning device. However, it will be appreciated that the invention is not limited to use with endoscopes or, more generally, to only use with medical devices. As such, it is to be appreciated that the techniques presented herein can be used to in association with the cleaning of lumens of a number of different devices/instruments used in any of a number of different applications, such as dental lines, food/drink lines, other medical lumens, etc. In addition, also as noted above, aspects of the techniques presented herein can also be used with manual lumen cleaning and, as such, reference to automatic lumen cleaning with a fluidic composition and/or a lumen cleaning device is merely illustrative.

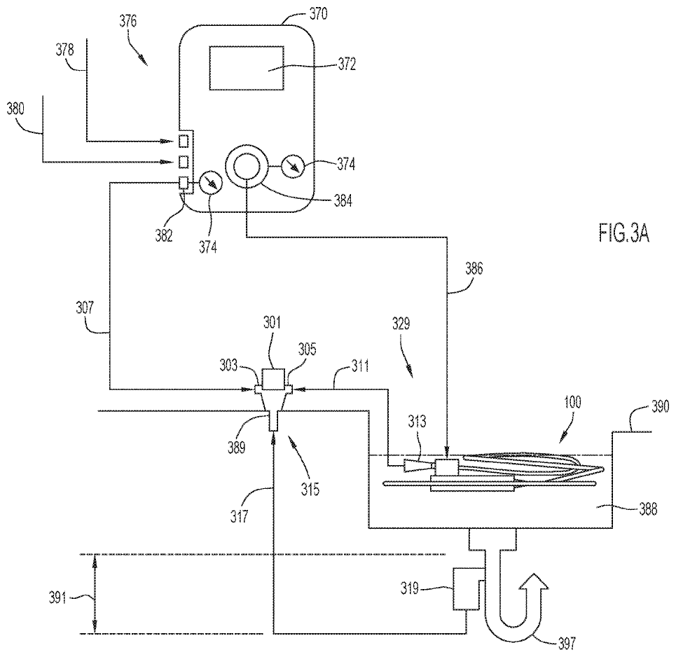

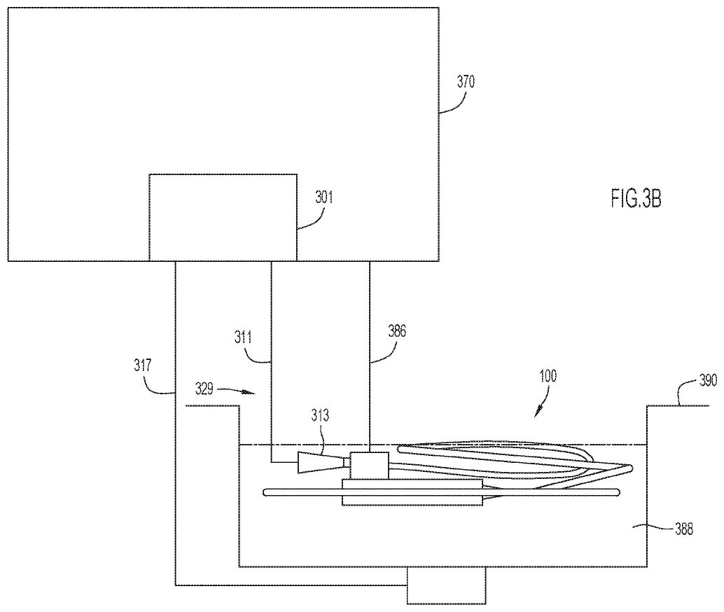

FIG. 3A is a schematic diagram illustrating management of a fluidic cleaning effluent with an effluent management accessory 301, in accordance with certain embodiments presented herein. More specifically, FIG. 3A illustrates an automated lumen cleaning device 370 (apparatus), which comprises a user interface 372, pressure sensors 374, and a plurality of connectors 376. The connectors 376 facilitate connection of the automated lumen cleaning device 370 to an air supply 378 (e.g., compressed dry air supply) and a water supply 380 (e.g., potable water supply). The connectors 376 also include a device outlet port 382.

As shown, the automated lumen cleaning device 370 also includes an interface/connector 384 for an endoscope adapter hose 386. The endoscope adapter hose 386 connects the automated lumen cleaning device 370 to one or more lumens of an endoscope, such as endoscope 100. During the exemplary cleaning process of FIG. 3A, the endoscope 100 can be substantially submerged under water 388 within, for example, a sink assembly 390. The sink assembly 390 includes a sink drain 397 (e.g., a P-trap drain).

As noted, also shown in FIG. 3A is the effluent management accessory 301 which, in this example, is fluidically connected to each of the automated lumen cleaning device 370 and the endoscope 100. More specifically, and as described in greater detail below, the effluent management accessory 301 includes two input ports, referred to as input port 303 input port 305. The input port 303 is fluidically connected to the device outlet port 382 via device outlet tubing/hose 307, while the input port 305 is fluidically connected to a distal end of one or more lumens of the endoscope 100 via an endoscope adapter tubing assembly 329. The endoscope adapter tubing assembly 329 includes, among other elements, tubing/hose 311 and an adapter 313. In certain examples, adapter 313 has a customer replaceable elastomer part that seals the endoscope tip. The effluent management accessory 301 also includes a drain port 315 connected to a drain assembly 317 that terminates in a spigot adapter 319 connected to the sink drain 397. As detailed further below, the drain assembly 317 may have a predetermined dip height 391.

As noted, FIG. 3A shows the effluent management accessory 301 fluidically connected to each of the automated lumen cleaning device 370 and the endoscope 100. In another arrangement, the effluent management accessory 301 could be fluidically connected to only the endoscope 100, and not to the automated lumen cleaning device 370.

The effluent management accessory 301 can be mounted to the sink assembly 390. In the illustrative example of FIG. 3A, the effluent management accessory 301 is mounted to the sink assembly 390 via a bore hole (with grommet) 389. In another alternative embodiment, as shown in FIG. 3B, the effluent management accessory 301 can also be integrated inside the automated lumen cleaning device 370.

As described further below, the effluent management accessory 301 operates by receiving fluidic cleaning effluent (fluidic effluent) from either the endoscope 100 (via adapter 313, hose 311, and input port 305) or directly from the automated lumen cleaning device 370 (via adapter 309, hose 307, and input port 303). The effluent management accessory 301 is configured to separate any gases (e.g., air) present in the fluidic effluent from any non-gases (e.g., liquids and/or solids) present in the effluent being discharged by an automatic or manual cleaning process. In general, the effluent management accessory 301 includes one or more separation stages (e.g., a first stage separator and, in certain examples, a second stage separator). In certain examples, a centrifugal separation stage or centrifugal separator (e.g., first stage separator) performs a centrifugal separation process to at least partially separate gas (e.g., air) portions of the fluidic effluent from non-gas portions of the fluidic effluent. The centrifugal separation stage (first stage separator) produces a “gaseous discharge” and a “non-gaseous discharge.” As used herein, the “non-gaseous discharge” is generally comprised of the liquid and/or solid portions of the fluidic effluent, such as water, blood, biofilms, contaminants, etc., but can also include some gas portions of the fluidic effluent. As used herein, the “gaseous discharge” is generally comprised of the gas portions of the fluidic effluent, but can also include relatively smaller amounts of liquid and/or solid portions of the fluidic effluent. In accordance with embodiments presented herein, the volume of solids/liquids is greater in the non-gaseous discharge than in the gaseous discharge. For example, in some embodiments, the volume of solids/liquids is greater in the non-gaseous discharge than in the gaseous discharge by at least 10%. In some embodiments, the gaseous discharge is greater than fifty percent (%) gas(es) by volume, whereas the non-gaseous discharge is greater than 50% liquid/solid by volume. In some embodiments, the volume of solids/liquids in the gaseous discharge is less than 50% of the volume of the gaseous discharge. In some embodiments, the volume of solids/liquids in the gaseous discharge is less than 25% of the volume of the gaseous discharge. In some embodiments, the volume of solids/liquids in the gaseous discharge is less than 10% of the volume of the gaseous discharge.

In accordance with embodiments presented herein, the non-gaseous discharge is discharged in the sink drain 397. However, in accordance with certain embodiments presented herein, a “refinement” or “second separation” stage (e.g., a second stage separator in the form of a filter cartridge assembly, a circuitous path, etc.), is provided to further refine the gaseous discharge produced in the centrifugal separation stage. That is, as described further below, the gaseous discharge is refined to further separate the remaining liquid and/or solid portions of the fluidic effluent from the remaining gas portions so that a “refined gaseous discharge” can be safely discharged to the room. That is, the refinement stage receives the gaseous discharge from the centrifugal separation stage and operates to further separate gas portions of the gaseous discharge from non-gas portions of the gaseous discharge, resulting in the release/discharge of a refined gaseous discharge into the environment (room). As used herein, a “refined gaseous discharge” is a gaseous discharge, produced via a centrifugal separation process, that has been secondarily refined with a refinement stage, such as a filter cartridge assembly, a circuitous path, etc.

The effluent management accessory 301 is operable to manage multi-fluid phase conditions, such as a mixed flow of gas, liquid, and solid, mixed flow of gas and liquid, gas only, liquid only, etc. That is, as used herein, the fluidic effluent can include a mixed flow of gas, liquid, and solid, mixed flow of gas and liquid, gas only, liquid only, etc. Certain design features are provided to manage these complicated multi-fluid phase conditions.

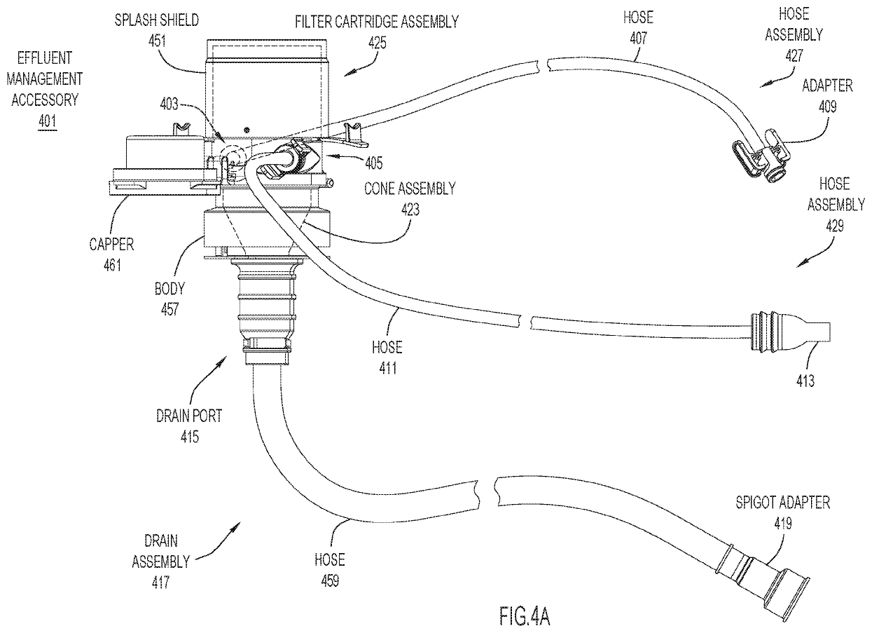

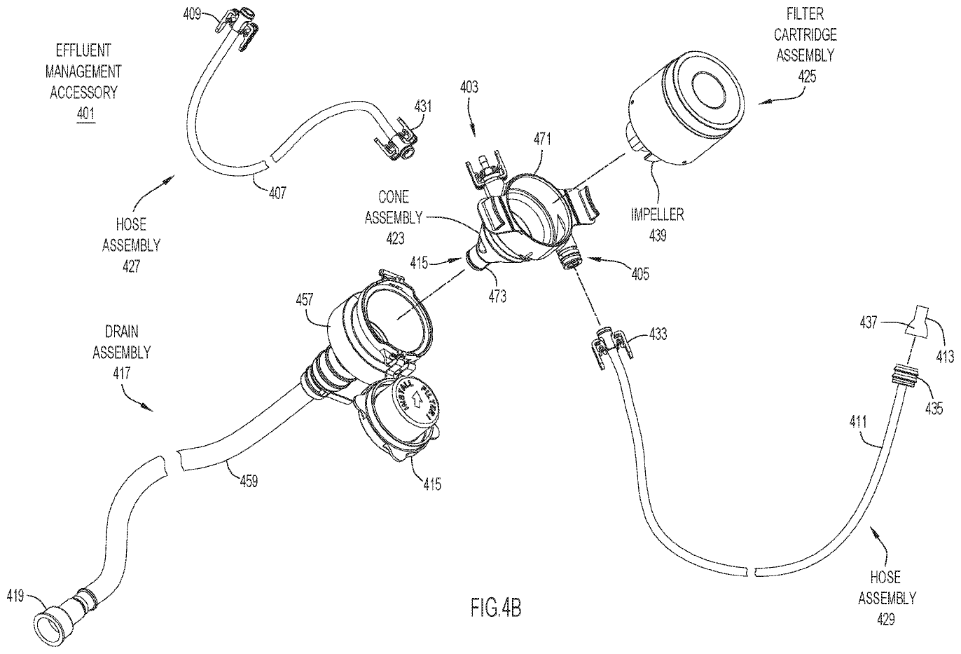

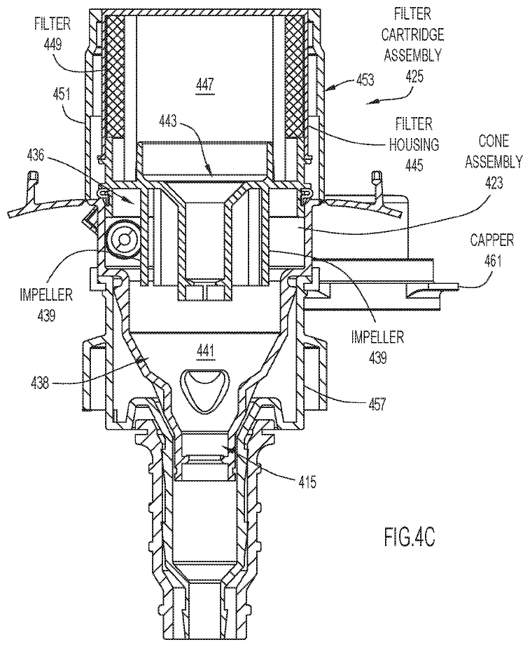

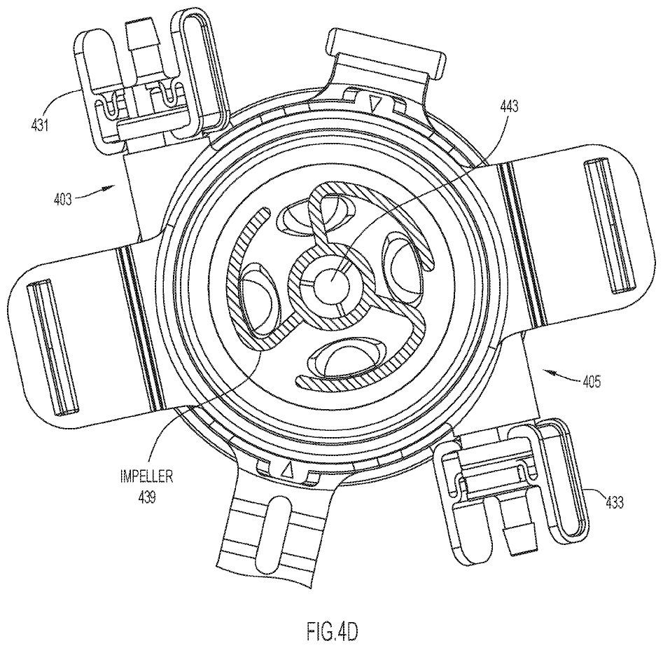

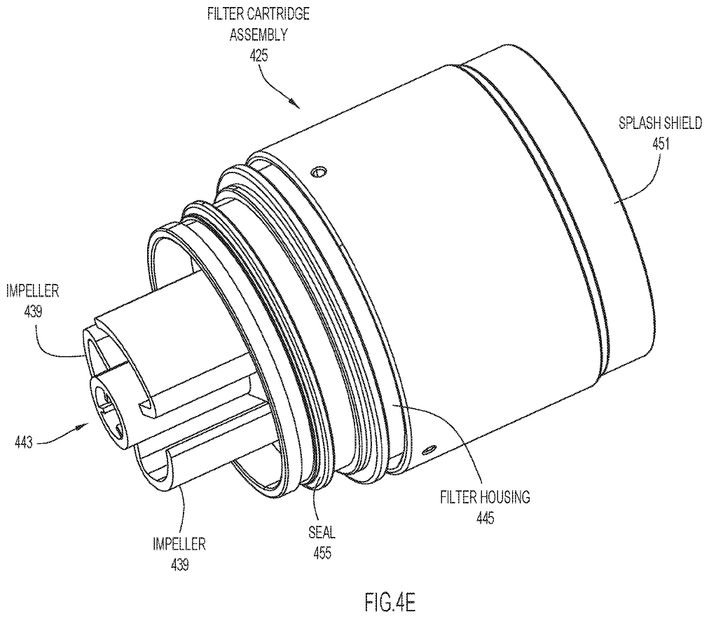

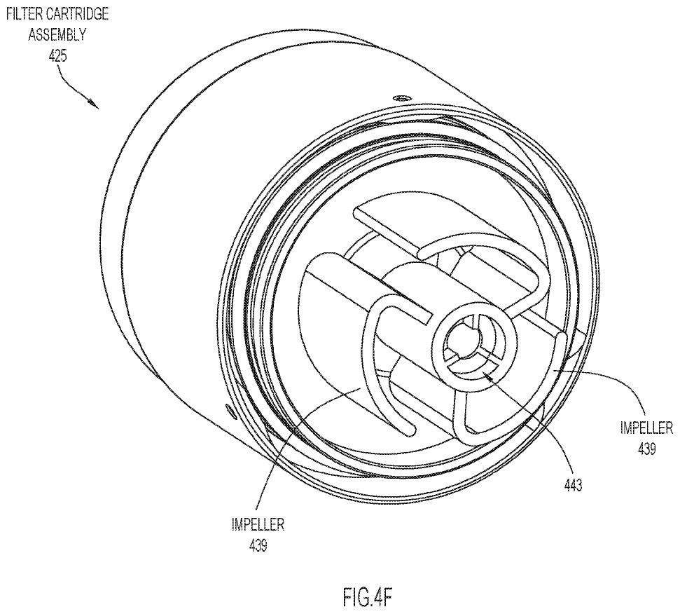

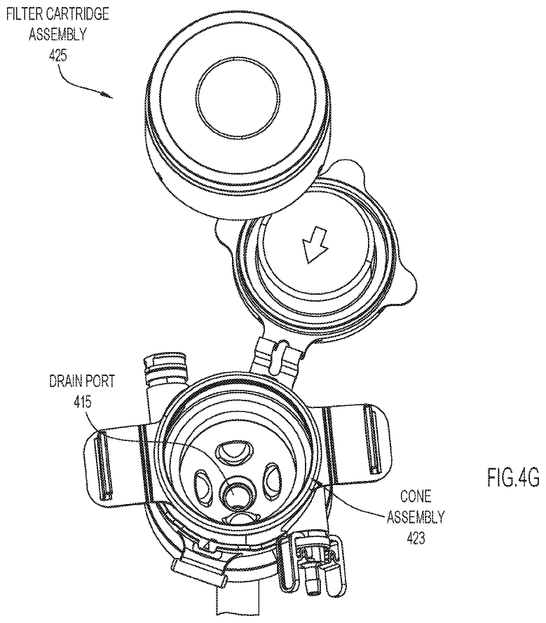

FIGS. 4A-4G are diagrams illustrating an example effluent management accessory 401, in accordance with certain embodiments presented herein, provided with both a centrifugal separation stage (e.g., first stage separator in the form of a centrifugal separator) and a refinement stage (e.g., second stage separator) in the form of a “filtration separation” stage. As described elsewhere herein, the presence of both the centrifugal separation stage and the filtration separation stage is merely illustrative. For example, it is to be appreciated that the techniques presented herein could be implemented with a device that omits the filtration separation stage. More specifically, FIG. 4A is a side view of the effluent management accessory 401, FIG. 4B is a partially-exploded view of the effluent management accessory 401, FIG. 4C is a first cross-sectional view of the effluent management accessory 401 in an operational configuration, and FIG. 4D is a second cross-sectional view of the effluent management accessory 401 in the operational configuration. FIGS. 4E and 4F are perspective view of a filter cartridge assembly of the effluent management accessory 401, while FIG. 4G is a top view of the effluent management accessory 401 in a non-operational configuration (i.e., with the filter cartridge assembly removed). For ease of explanation, FIGS. 4A-4G will generally be described together.

The effluent management accessory 401 is described as generally comprising five (5) portions/sections, referred to as a drain assembly 417, a cone assembly 423, a filter cartridge assembly 425, a first hose/tube assembly 427, and a second hose/tube assembly 429. Each of these portions will be described in greater detail below. However, is to be appreciated that the general division of the effluent management accessory 401 into these specific five portions is merely for ease of description and that, in alternative arrangements, effluent management accessories presented herein can include different numbers of portions having a number of different structural arrangements.

Similar to effluent management accessory 301 of FIGS. 3A and 3B, the effluent management accessory 401 operates by receiving fluidic cleaning effluent (fluidic effluent), which can comprise multi-fluid phase conditions, such as a mixed flow of gas, liquid, and solid, mixed flow of gas and liquid, gas only, liquid only, etc. As described further below, the effluent management accessory 401 performs at least two main functions in relation to a received effluent, including both a centrifugal separation and a filtration separation of gases from any liquids and/or solids present in the fluidic effluent. That is, the effluent management accessory 401 is configured to separate gases (e.g., air) present in the fluidic cleaning effluent from any liquids and solids present in the effluent being discharged by an automatic or manual cleaning process.

The centrifugal separation stage uses a centrifugal separation process to substantially separate liquids and solid portions (particles) of the fluidic effluent from gas portions of the fluidic effluent to producing a gaseous discharge and a non-gaseous discharge. However, as described further below, the filtration separation stage is applied to only the gaseous discharge (e.g., the separated substantially gas portions produced during the centrifugal separation stage), while the non-gaseous discharge (e.g., relatively larger liquids and solid portions of the fluidic effluent) is immediately discarded. In the embodiment of FIGS. 4A-4G, the filtration separation stage uses a filtration process (e.g., coalescing filter) to separate liquid and solid portions (particles) of the fluidic effluent from gas portions of the fluidic effluent. As such, only the gas portions of the fluidic effluent are safely discharged to the room, while liquid and solid effluent are safely discharged into, for example, a sink drain. The structural arrangement of the effluent management accessory 401 is described further below, followed by a more detailed explanation of the functional operation of the effluent management accessory 401.

The effluent management accessory 401 first comprises the cone assembly 423 having two input ports, referred to as input port 403 and input port 405, that are each configured to receive fluidic effluent from one or more fluidic effluent sources. In this example, the input port 403 is configured to be fluidically connected to a drain fitting/port (not shown in FIGS. 4A-4G) of a lumen cleaning device (e.g., endoscope reprocessing device's drain port) via the first hose assembly 427, while the input port 405 is configured to be fluidically connected to a distal end (not shown in FIGS. 4A-4F) of one or more lumens (e.g., distal end of an endoscope)

The hose assembly 427 comprises an adapter 409 for connection to the drain port of the lumen cleaning device, a connector 431 (e.g., quick disconnect) for connection to the input port 403, and a hose/tube 407 fluidically connecting the adapter 409 to the connector 431. The hose assembly 429 comprises an adapter 413 for connection to the distal end of one or more lumens, a connector 433 (e.g., quick disconnect) for connection to the input port 405, and a hose/tube 411 fluidically connecting the adapter 413 to the connector 433. In this example, the adapter 412 is configured to be fitted into a connector 435, and includes a backflow arrestor 437 (backflow minimizer). In this example, the backflow arrestor 437 comprises a cone-shaped piece that minimizes the flow of effluent back into the hose 411. It is to be appreciated that this specific arrangement for the backflow arrestor 437 is merely illustrative and that other types of backflow arrestors could be used in alternative embodiments.

As noted, the effluent management accessory 401 of FIGS. 4A-4G includes two input ports that can receive effluent. It is to be appreciated that the presence of two input ports is merely illustrative and that other embodiments can include a single input port, or more than two input ports.

Returning to the specific example of FIGS. 4A-4G, the cone assembly 423 is an element of the centrifugal separation stage and is the interface for the drain port of the lumen cleaning device, the lumen(s) being cleaned, and the sink drain path. As shown, at least part of the cone assembly 423 defines a general conical or tapered internal volume 441 that terminates in a drain port 415. In the examples of FIGS. 4A-4G, the cone assembly 423 includes a generally cylindrical upper portion 436, where the ports 403 and 405 are located, and a lower portion 438 with a conical volume. In other embodiments, the upper portion could be omitted such that the entire cone assembly 423 defines a general conical internal volume.

Disposed within the internal volume of the cone assembly 423 are baffles/separator blades 439 (impeller) having a central aperture 443 (a through-hole), sometimes referred to as a “vortex finder.” In the example of FIGS. 4A-4G, the baffles 439 are part of the filter cartridge assembly 425. However, in alternative embodiments, the baffles 439 could be separate from the filter cartridge assembly 425.

In certain examples, the cone assembly 423 is referred to as having a first end 471 and a second end 473. As shown, the baffles 439 are disposed in the cone assembly 423 adjacent the first end 471, while the drain port 415 is disposed at the second end 473. As such, the cone assembly 423 defines a substantially conical volume between the baffles 439 and the drain port 415.

In addition to the baffles 439, the filter cartridge assembly 425 defines an internal volume 447 that is disposed above the central aperture 443 of the baffles 439, where the central aperture 443 provides the only fluidic connection between the internal volume 441 and the internal volume 447. The internal volume 447 is circumferentially surrounded by a filter 449, which in turn is disposed in a filter housing 445. The filter housing 445 also includes openings 453, and a splash shield 451 may also be provided.

In certain embodiments, the filter cartridge assembly 425 is secured to the cone assembly 423 (e.g., by two cantilever flaps). In alternative embodiments, the filter cartridge assembly 425 could be secured to the cone assembly 423 in a different manner, secured to the drain assembly 417, etc. A seal 455 (e.g., O-ring) can be provided between the filter cartridge assembly 425 and the cone assembly 423.

As shown most clearly shown in FIG. 4B, the drain assembly 417 includes a body or cradle assembly 457 that is configured to receive and secure the cone assembly 423. The body 457 can swivel to accommodate different device-to-sink orientations. The drain assembly 417 also includes a drain hose/tube 459 having a first end connected to the body 457 and a second end connected to a spigot adapter 419. The drain port 415 of the cone assembly 423 is fluidically connected to the first end of the drain hose 459 and the spigot adapter 419 is configured to be connected to a sink drain (not shown in FIGS. 4A-4F). That is, the spigot adapter 419 is used to securely connect the drain hose to a sink drain, such as a P-trap. The spigot adapter 419 can be, in certain embodiments, universal for all global standard spigot sizes.

Shown attached to the body 457 is a capper 461. The capper 461 is configured to mate with the body 457 when the filter cartridge assembly 425 is not installed therein to minimize the chance that the effluent management accessory 401 is operated without the filter cartridge. When inserted into the body 457, the capper 461 physically blocks the input ports 403 and 405.

As noted above, the effluent management accessory 401 is configured to perform dynamic separation of gas (e.g., air) from the liquid and solid effluent in two stages. The centrifugal separation stage occurs within the cone assembly 423, while the filtration separation stage occurs within the filter cartridge assembly 425. In the centrifugal separation stage, the received effluent is impacted along the radial inside wall of the cone assembly 423 and, due to high inertial (centrifugal) forces of liquid, water, and solid particles, the mixture will traverse downward towards the drain port 415, leaving the gases exiting towards the central aperture 443 (vortex finder) and into the internal volume 447 of the filter cartridge assembly 425. The baffles 439 is an integrated feature in the centrifugal separation stage that provides a boundary between the central aperture 443 and the air radial stream, while not disrupting the internal wall of flow vortices.

Stated differently, in the centrifugal separation stage, the fluidic effluent (received from the endoscope 100 and automated lumen cleaning device 370) is fed tangentially into the cylindrical top of the cone assembly 423 to create rotation. Passing through the cylindrical top where the flow velocity is further accelerated. The centrifugal force separates the stream of non-gas portions of the fluidic effluent while it rotate downward following the surface profile of cone assembly 423. While the stream of gas portions of the fluidic effluent are moved to enter the baffles 439 during the rotation.

In summary, the centrifugal separation stage (centrifugal separator) is used to at least partially separate the gas effluent from the solid and liquid effluent, thereby producing what is referred to herein as a “gaseous discharge” (e.g., separated and potentially moist gases) and a “non-gaseous discharge” (e.g., generally liquids and/or solids). The gaseous discharge enters into the central aperture 443, while the non-gaseous discharge (solid and liquid portions) passes to the drain port 415 (e.g., as a result of gravity) and, eventually, to the drain hose 459 and connected drain. Stated differently, the heavier non-gas particles are separated from the gas via centrifugal force, where the mass of the non-gas particles is larger and thus these larger particles impact the walls of the cone assembly 423 harder to separate them from the gas particles, where gravity pulls the non-gases down into the drain.

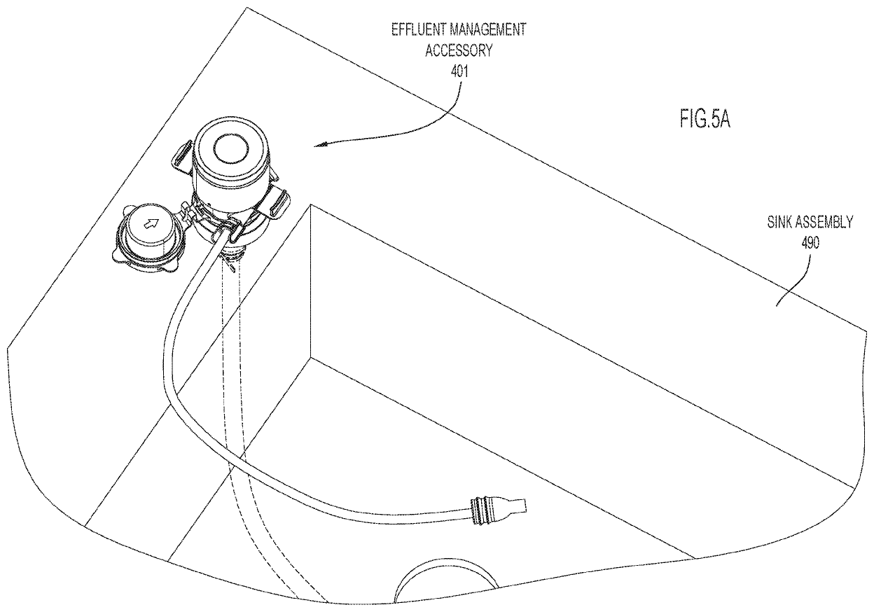

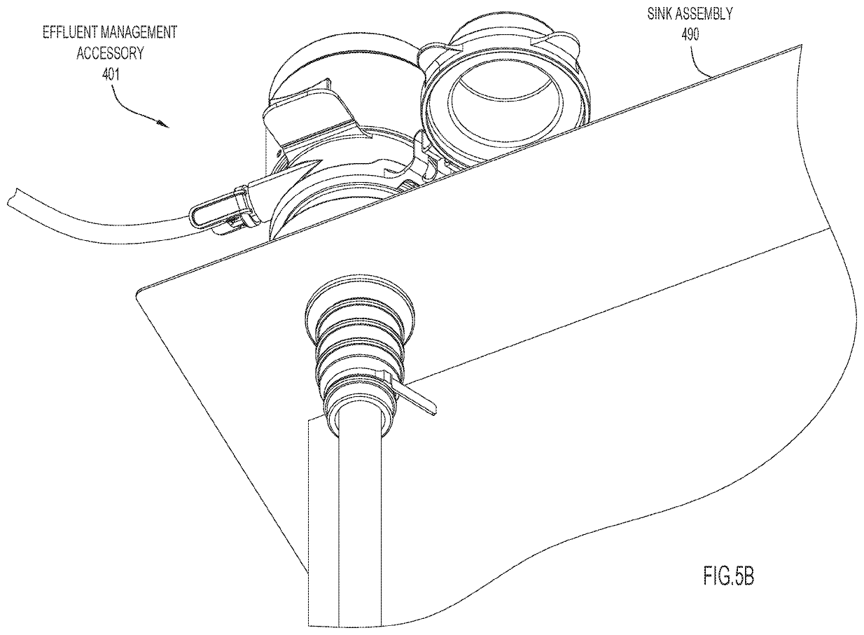

In certain examples, this centrifugal separation stage utilizes two conditions that are enabled by a physical arrangement of the effluent management accessory 401. In particular, during operation, the effluent management accessory 401 should be oriented such that the drain port 415 is positioned inferior to the baffles 439, thereby allowing gravity to pull the non-gaseous discharge (the heavier solids and liquids) down towards the drain port 415, while allowing the lighter gaseous discharge to enter the central aperture 443. FIGS. 5A and 5B are top and bottom perspective views, respectively, illustrating the effluent management accessory 401 mounted to a sink assembly 490 in an orientation enabling gravity to pull the heavier non-gaseous discharge down towards the drain port 415 and enabling the gaseous discharge to enter the central aperture 443. It is noted that, in FIG. 5B, a portion of the sink assembly 490 has been omitted to more clearly illustrate aspects of the effluent management accessory 401.

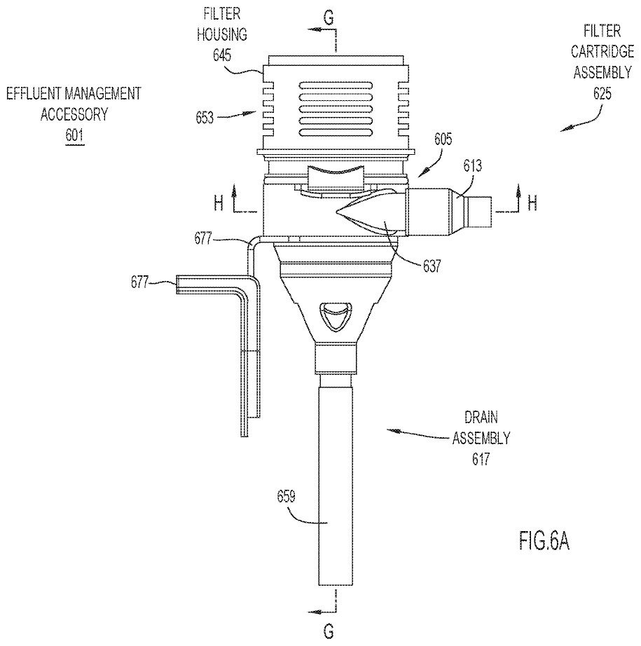

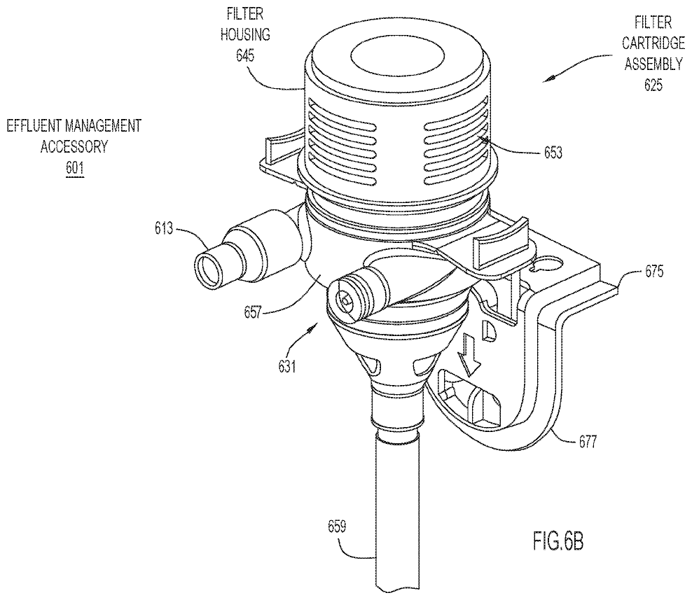

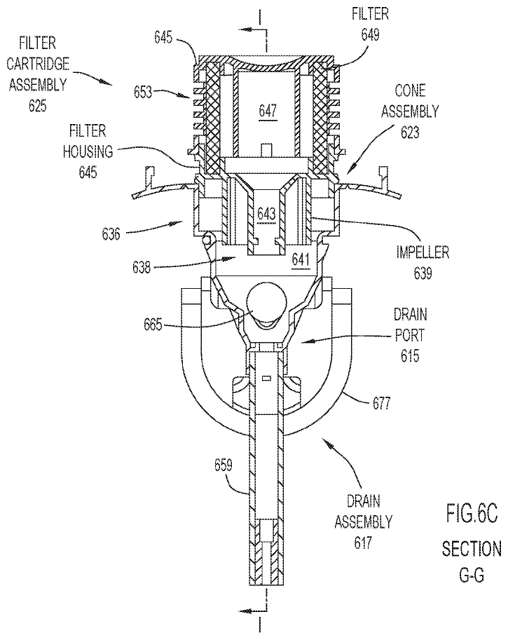

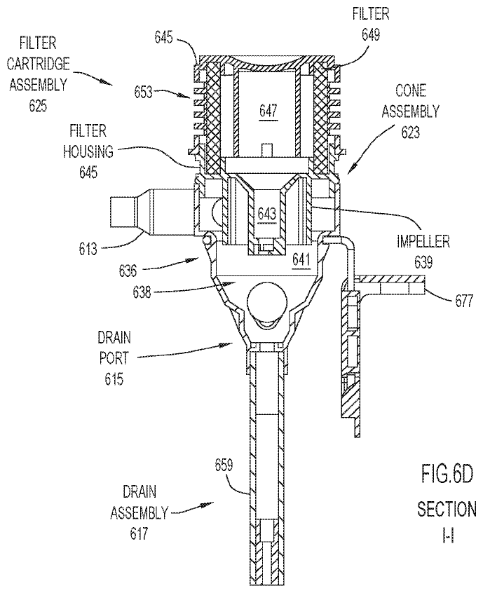

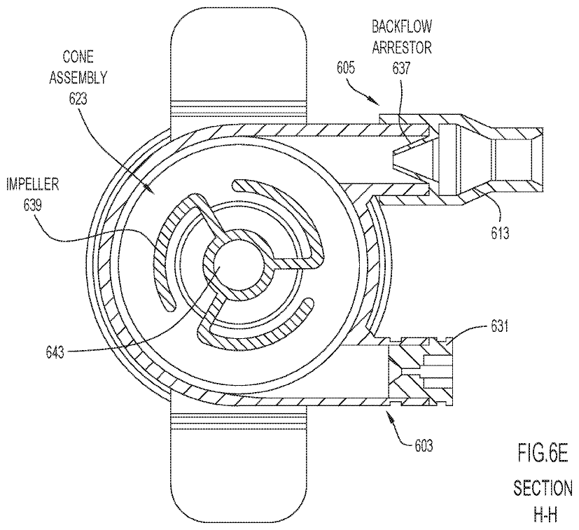

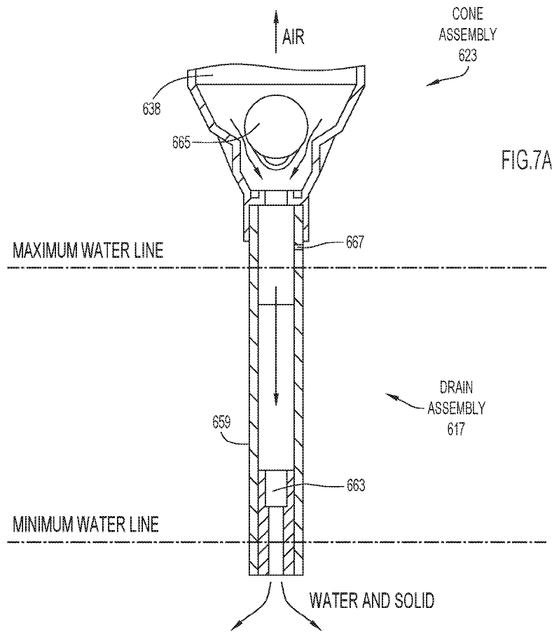

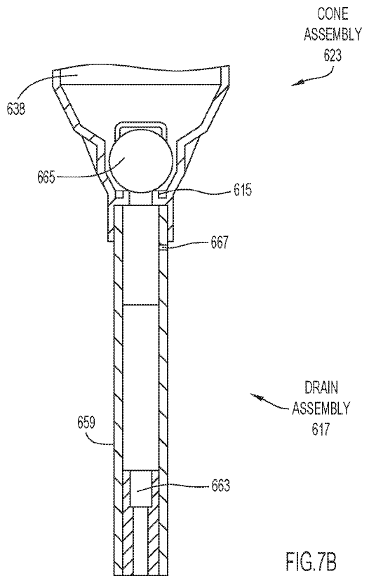

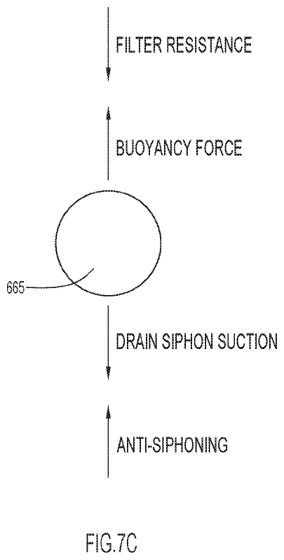

In addition to an upright orientation of the effluent management accessory 401, a second physical condition during operation is that the fluidic resistance of the drain hose 459 should be greater than the fluidic resistance of the filter cartridge assembly 425, particularly with a gas only effluent flow (e.g., during air purging). If the fluidic resistance of the filter cartridge assembly 425 is greater than the fluidic resistance of the drain hose, then gaseous discharge could escape through the drain hose 459 and the connected drain, resulting in aerosolization. The relatively greater fluidic resistance of the drain hose 459 could be provided, for example, by the inclusion of a dynamic shut-off valve (e.g., a ball valve as shown in FIGS. 6A-6E and 7A-7C) at the drain port 415, or by providing a minimum water column (dip height) within the drain hose 459 (e.g., refer to predetermined dip height 391 as shown in FIG. 3A).

As noted, the centrifugal separation stage is used to at least partially separate the gas effluent from the solid and liquid effluent. In certain examples, as noted above, the gaseous discharge (e.g., gas effluent and remaining relatively smaller liquid or solid particles) enters the central aperture 443 (vortex finder), and passes into the internal volume 447 of the filter cartridge assembly 425. As noted, the filter cartridge assembly 425 (filter housing 445) includes openings 453, where the filter 449 is located between the openings and the central aperture 443. As such, the gaseous discharge passes from the internal volume 447 to the filter 449, which separates the remaining relatively smaller liquid or solid particles from the remaining gas(es). As such, the remaining gas(es) can pass through the filter 449, producing a refined gaseous discharge that can exit through the openings 453 to the ambient environment (e.g., by passing around the splash shield 451), while the remaining relatively smaller liquid or solid particles are trapped by the filter 449. The filter 449 can remove excessive water vapor from the gas to prevent any dramatic increase of moisture within the ambient environment.

In summary, the filter cartridge assembly 425 manages the solid and liquid particles that were not collected by the centrifugal separation stage. In general, these particles have a size that is below the “cut-off” diameter of the centrifugal separation stage Collection Efficiency Threshold (CET). As described elsewhere herein, the filtration separation stage (or other type of refinement stage) can be implemented in different manner or, in certain embodiments, a centrifugal separation stage operates alone to manage a fluidic effluent (e.g., the filtration separation stage is omitted).

In certain embodiments, the filter 449 may be a coalescing type of filter with relatively low pressure drop characteristics and high collection efficiency (e.g., 0.1~0.30 um rating). In specific examples, the filter 449 is formed from a Borosilicate Microfiber material. In certain embodiments, the filter 449 can be, for example, replaceable and/or can cleaned via back-pressure. In certain embodiments, the entire filter cartridge assembly 425 is a consumable/disposable component, whereas the other components (e.g., cone assembly, drain assembly, etc.) can be cleaned for re-use.

FIGS. 4A-4G generally illustrate one example arrangement for an effluent management accessory, in accordance with certain embodiments presented herein. It is to be appreciated that the arrangement shown in FIGS. 4A-4G are merely illustrative and that effluent management accessories in accordance with embodiments presented herein can be implemented with different arrangements. For example, FIGS. 6A-6E illustrate another arrangement for an effluent management accessory, referred to as effluent management accessory 601, in accordance with certain embodiments presented herein.

More specifically, FIG. 6A is a side view of the effluent management accessory 601 and FIG. 6B is a perspective view of effluent management accessory 601. FIG. 6C is a first cross-sectional view of the effluent management accessory 601 (taken along line G-G in FIG. 6A), FIG. 6D is a second cross-sectional view of the effluent management accessory 601 (taken along line I-I in FIG. 6A), and FIG. 6E is a third cross-sectional view of the effluent management accessory 601 (taken along line H-H in FIG. 6A).

The effluent management accessory 601 generally comprises a drain assembly 617, a cone assembly 623, and a filter cartridge assembly 625. Each of these portions will be described in greater detail below. However, is to be appreciated that the general division of the effluent management accessory 601 into these specific portions is merely for ease of description and that, in alternative arrangements, effluent management accessories presented herein can include different numbers of portions having a number of different structural arrangements.

The effluent management accessory 601 operates by receiving fluidic cleaning effluent (fluidic effluent), which can comprise multi-fluid phase conditions, such as a mixed flow of gas, liquid, and solid, mixed flow of gas and liquid, gas only, liquid only, etc. As described further below, the effluent management accessory 601 performs at least two main functions in relation to a received fluidic effluent, including a centrifugal separation stage to substantially separate gases from any liquids and/or solids, producing a gaseous discharge and a non-gaseous discharge, and a filtration separation stage separation of the gaseous discharge. That is, the effluent management accessory 601 is configured to separate any gases (e.g., air) present in the fluidic cleaning effluent from any liquids and solids present in the effluent being discharged by an automatic or manual cleaning process. The separated (moist) gases (gaseous discharge) are refined (e.g., filtered) so that they can be safely discharged to the room, while liquid and solid effluent can be safely discharged into, for example, a sink drain. The structural arrangement of the effluent management accessory 601 is described further below, followed by a more detailed explanation of the functional operation of the effluent management accessory 601.

The effluent management accessory 601 first comprises the cone assembly 623 having two input ports, referred to as input port 603 and an input port 605, that are configured to receive effluent. That is, the input port 603 and the input port 605 are each configured to be fluidically connected to a fluidic cleaning effluent source. In this example, the input port 603 is configured to be fluidically connected to a drain fitting/port (not shown in FIGS. 6A-6E) of a lumen cleaning device (e.g., endoscope reprocessing device's drain port), while the input port 605 is configured to be fluidically connected to a distal end (not shown in FIGS. 6A-6E) of one or more lumens (e.g., distal end of an endoscope).

In the examples of FIGS. 6A-6E, an adapter 613 for connection to the distal end of one or more lumens is attached to the input port 605. The adapter 613 includes a backflow arrestor 637. In this example, the backflow arrestor 637 comprises a cone-shaped piece that minimizes the flow of effluent back into the lumens. In FIG. 6A, a portion of the outer surface of the effluent management accessory 601 has been omitted to show the backflow arrestor 637.

It is to be appreciated that this specific arrangement for the backflow arrestor 637 is merely illustrative and that other types of backflow arrestors could be used in alternative embodiments. In addition, a connector 631 (e.g., quick disconnect) is disposed at the input port 603. The connector 631 is operate for connection to a hose/tube (not shown in FIGS. 6A-6E) which, in turn, can be connected to the lumen cleaning device.

As noted, the effluent management accessory 601 of FIGS. 6A-6E includes two input ports that can receive effluent. It is to be appreciated that the presence of two input ports is merely illustrative and that other embodiments can include a single input port, or more than two input ports. In various embodiments, the input ports 603 and 605 can be configured for direct connection to the appropriate fluidic cleaning effluent source, or may be each be configured for connection to the appropriate fluidic cleaning effluent source via a hose assembly that may be similar to, or different from, the hose assemblies described elsewhere herein.

Returning to the specific example of FIGS. 6A-6E, the cone assembly 623 is an element of the centrifugal separation stage and is the interface for the drain port of the lumen cleaning device, the lumen(s) being cleaned, and the sink drain path. As shown, at least part of the cone assembly 623 defines a general conical or tapered internal volume 641 that terminates in a drain port 615. In the examples of FIGS. 6A-6E, the cone assembly 623 includes a generally cylindrical upper portion 636, where the input ports 603 and 605 are located, and a lower portion 638 with the conical volume. In other embodiments, the upper portion could be omitted such that the entire cone assembly 623 defines a general conical internal volume.

Disposed within the internal volume of the cone assembly 623 are baffles/separator blades 639 (impeller) having a central aperture 643 (a through-hole), sometimes referred to as a “vortex finder.” In the example of FIGS. 6A-6E, the baffles 639 are part of the filter cartridge assembly 625. However, in alternative embodiments, the baffles 639 could be separate from the filter cartridge assembly 625.

In addition to the baffles 639, the filter cartridge assembly 625 comprises a filter cover 645 defining an internal volume 647 that is disposed above the central aperture 643 of the baffles 639. The internal volume 647 is circumferentially surrounded by a filter 649, which in turn is covered by the filter cover 645 (e.g., to seal the filter 649 end and to protect the filter from water splashes and accidental damage). The filter cover 645 also includes openings 653.

In certain embodiments, the filter cartridge assembly 625 is secured to the cone assembly 623 (e.g., by two cantilever flaps). In alternative embodiments, the filter cartridge assembly 625 could be secured to the cone assembly 623 in a different manner, secured to the drain assembly 617, etc. A seal (e.g., O-ring) can be provided between the filter cartridge assembly 625 and the cone assembly 623.

The drain assembly 617 includes a body or cradle assembly 657 that is configured to receive and secure the cone assembly 623. In certain examples, a cradle ring 675 is provided to hold the effluent management accessory 601 upright. The cradle ring 675 is operable to swivel, and is attached to a cradle base 677 that can be anchored to a surface, such as a sink assembly surface (e.g., double-sided adhesives or screws). The drain assembly 617 also includes a drain tube/hose 659, which in turn includes a drain orifice 663 that is configured to regulate discharge flow.

As noted above, the effluent management accessory 601 is configured to perform dynamic separation of gas (e.g., air) from the liquid and solid effluent in two stages. The centrifugal separation stage occurs within the cone assembly 623, while the filtration separation stage occurs within the filter cartridge assembly 625. In the centrifugal separation stage, the received effluent is impacted along the radial inside wall of the cone assembly 623 and, due to high inertial (centrifugal) forces of liquid, water, and solid particles, the mixture will traverse downward towards the drain port 615, leaving the gases exiting towards the central aperture 643 (vortex finder) and into the internal volume 647 of the filter cartridge assembly 625. The baffles 639 are an integrated feature in the centrifugal separation stage that provides a boundary between the central aperture 643 and the air radial stream, while not disrupting the internal wall of flow vortices.

In summary, the centrifugal separation stage (centrifugal separator) is used to at least partially separate the gas effluent from the solid and liquid effluent, thereby producing what is referred to herein as a “gaseous discharge” (e.g., separated and potentially moist gases) and a “non-gaseous discharge” (e.g., liquids and/or solids). The gaseous discharge enters the central aperture 643, while the non-gaseous discharge (solid and liquid portions) passes to the drain port 615 (e.g., as a result of gravity) and, eventually, to the drain hose 659 and connected drain.

In certain examples, this centrifugal separation stage utilizes two conditions that are enabled by a physical arrangement of the effluent management accessory 601. In particular, during operation, the effluent management accessory 601 should be oriented such that the drain port 615 is positioned inferior to the baffles 639, thereby allowing gravity to pull the non-gaseous discharge (heavier solid and liquid) down towards the drain port 615, while allowing the lighter gaseous discharge to enter the central aperture 643. In addition to an upright orientation of the effluent management accessory 601, a second physical condition during operation is that the fluidic resistance of the drain hose 659 should be greater than the fluidic resistance of the filter cartridge assembly 625, particularly with a gas only effluent flow (e.g., during air purging). If the fluidic resistance of the filter cartridge assembly 625 is greater than the fluidic resistance of the drain hose, then the gaseous discharge could escape through the drain hose 659 and the connected drain, resulting in aerosolization. The relatively greater fluidic resistance of the drain hose 659 is provided, in this example, by the inclusion of a dynamic shut-off valve 665 at the drain port 615. The illustrative dynamic shut-off valve 665 of FIGS. 6A-6E is a ball valve, which is further described below with reference to FIGS. 7A-7C.

As noted, the centrifugal separation stage is used to substantially separate the gas effluent from the solid and liquid effluent. In certain examples, as noted above, the gaseous discharge (e.g., gas effluent and remaining relatively smaller liquid or solid particles) enters the central aperture 643 (vortex finder), and passes into the internal volume 647 of the filter cartridge assembly 625. As noted, the filter 649 is located between the internal volume 647 and any exits to the ambient environment. As such, the gaseous discharge passes from the internal volume 647 to the filter 649, which separates the remaining relatively smaller liquid or solid particles from the remaining gas(es). As such, the remaining gas(es) can pass through the filter 649, producing a refined gaseous discharge that is released into the ambient environment, while the remaining relatively smaller liquid or solid particles are trapped by the filter 649. In certain examples, the filter 649 removes excessive water vapor from the gas to prevent any dramatic increase of moisture within the ambient environment.

In summary, the filter cartridge assembly 625 manages the solid and liquid particles that were not collected by the centrifugal separation stage. These particles have a size that is below the “cut-off” diameter of the centrifugal separation stage Collection Efficiency Threshold (CET). As noted above, in some embodiments, a filtration separation stage as described above, or another technique (e.g., a circuitous pathway), is further used to refine the fluidic effluent. In other embodiments, a centrifugal separation stage operates alone to manage a fluidic effluent.

In certain embodiments, the filter 649 may be a coalescing type of filter with relatively low pressure drop characteristics and high collection efficiency (e.g., 0.1~0.30 um rating). In specific examples, the filter 649 is formed from a Borosilicate Microfiber material. In certain embodiments, the filter 649 can be, for example, replaceable and/or can cleaned via back-pressure. In certain embodiments, the entire filter cartridge assembly 625 is a consumable/disposable component, whereas the other components (e.g., cone assembly, drain assembly, etc.) can be cleaned for re-use.

As noted above, the effluent management accessory 601 includes a cylindrical hollow ball valve 665 (e.g., hollow plastic ball acting as a shut-off valve) that operates to prevent the gas (air) from coming out through the drain hose 659 during, for example, an air purging operation (e.g., physically blocking the drain path with a gas only flow). As noted, FIGS. 7A-7C illustrate operation of the ball valve 665, where FIGS. 7A and 7B are cross-sectional views of a portion of the effluent management accessory 601 and FIG. 7C is a schematic illustration of the ball valve operation.

More specifically, as shown on FIG. 7A, the ball valve 665 disengages when both gas (air) and a liquid (water) are present inside the cone assembly. However, as shown in FIG. 7B, the ball valve 665 engages if there is a gas only effluent because the ball valve 665 does not float in gas (e.g., the ball valve 665 will float when there is water in the system, but prevents air from coming out into the drain pipe when water is not present).

The ball valve 665 function is regulated by a breather 667, the drain hose 659, and drain orifice 663. These three components balance the forces, as shown in FIG. 7C, acting on the ball valve 665 to prevent any lock-up. As such, in general, the buoyant cylindrical ball valve 665 acts as a valve to seal off when liquid is not present inside the assembly 623 and floats open when there is liquid inside the cone assembly. In certain examples, the ball valve 665 can allow a lumen cleaning device to detect if the back pressure at filter 669 is beyond acceptable limits, where increased filter resistance will can have a negative impact on the functionality and efficiency.

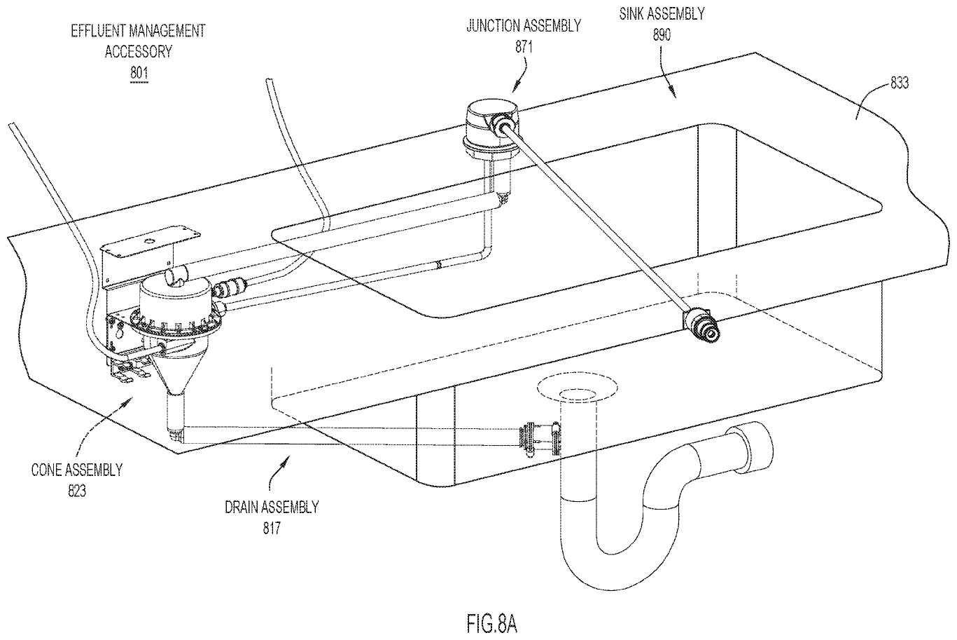

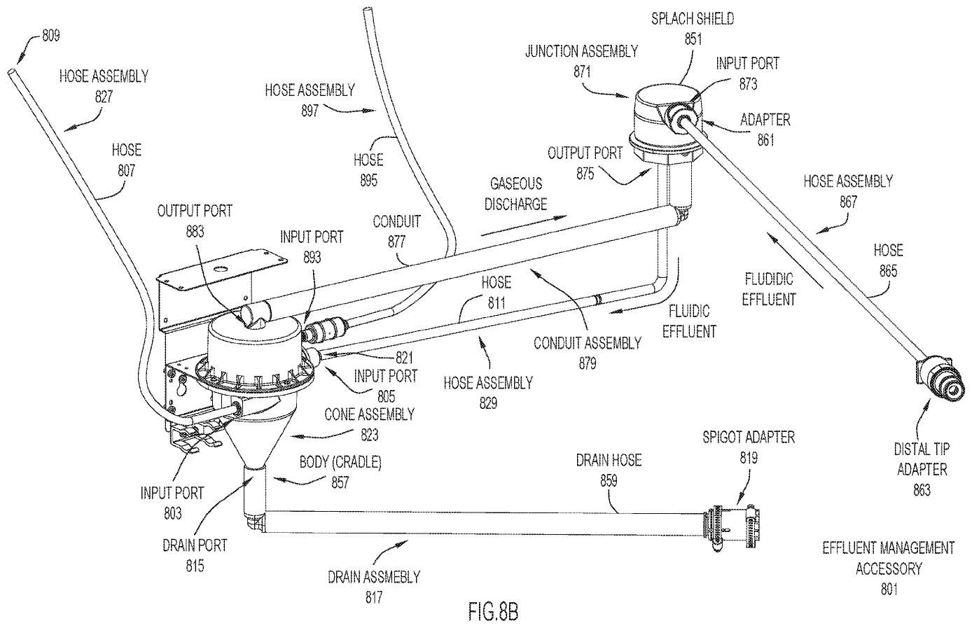

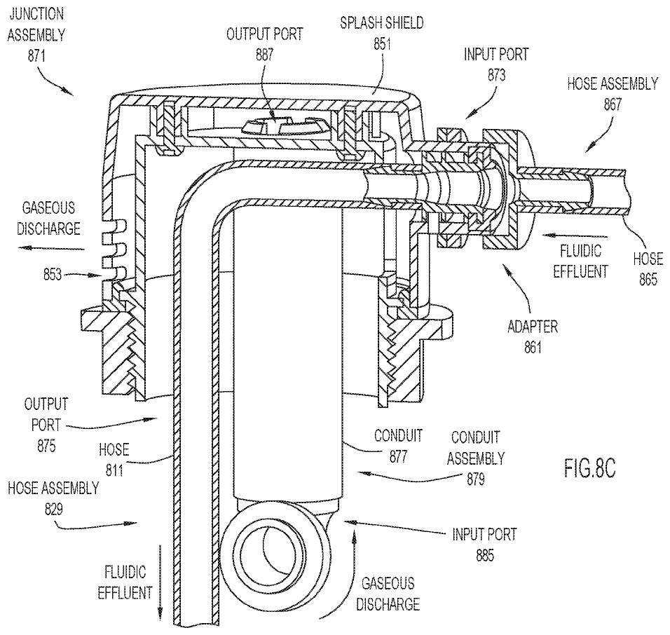

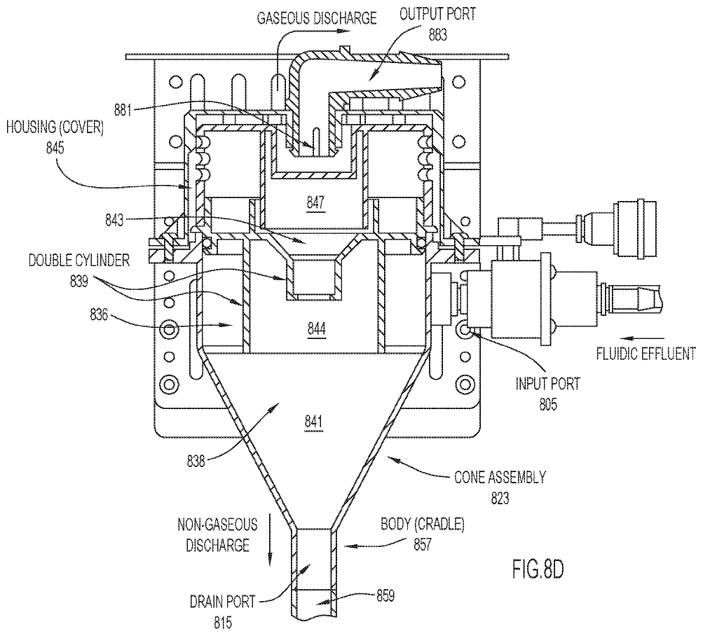

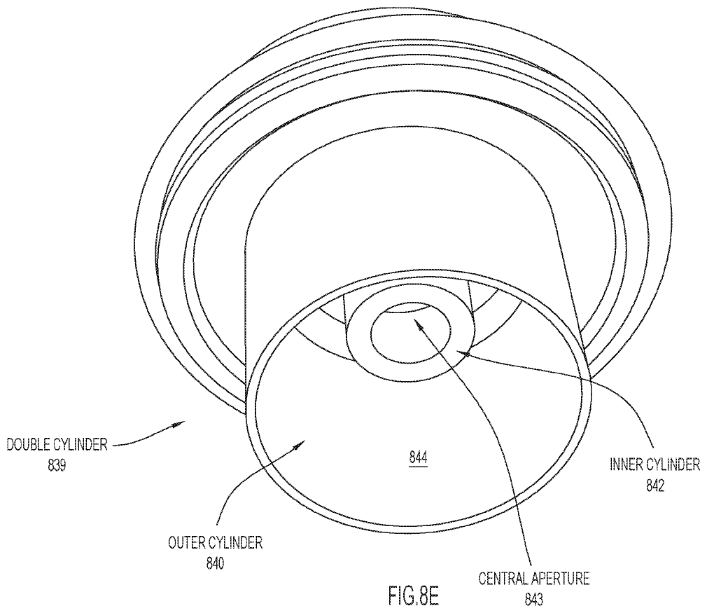

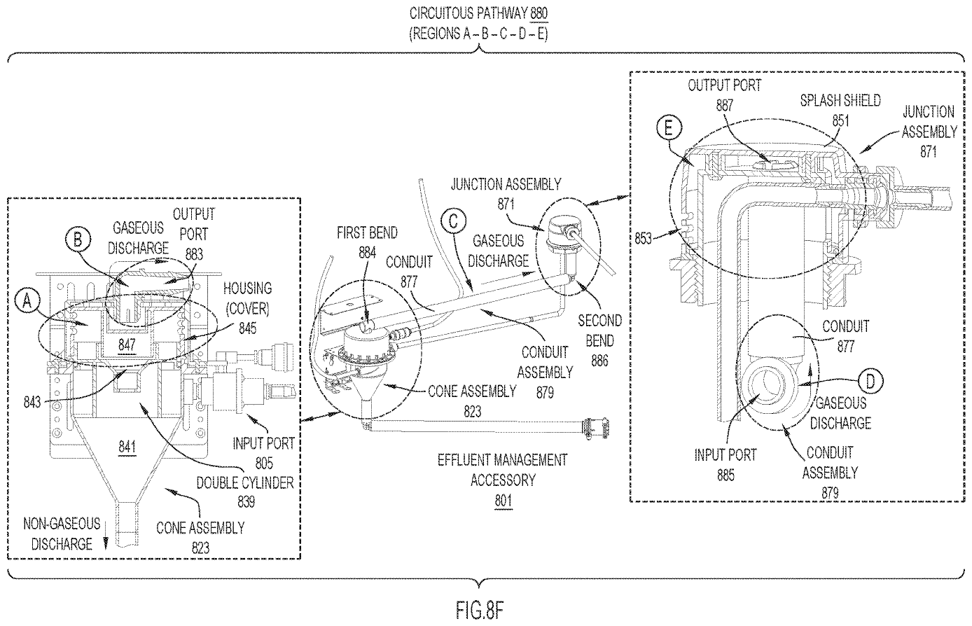

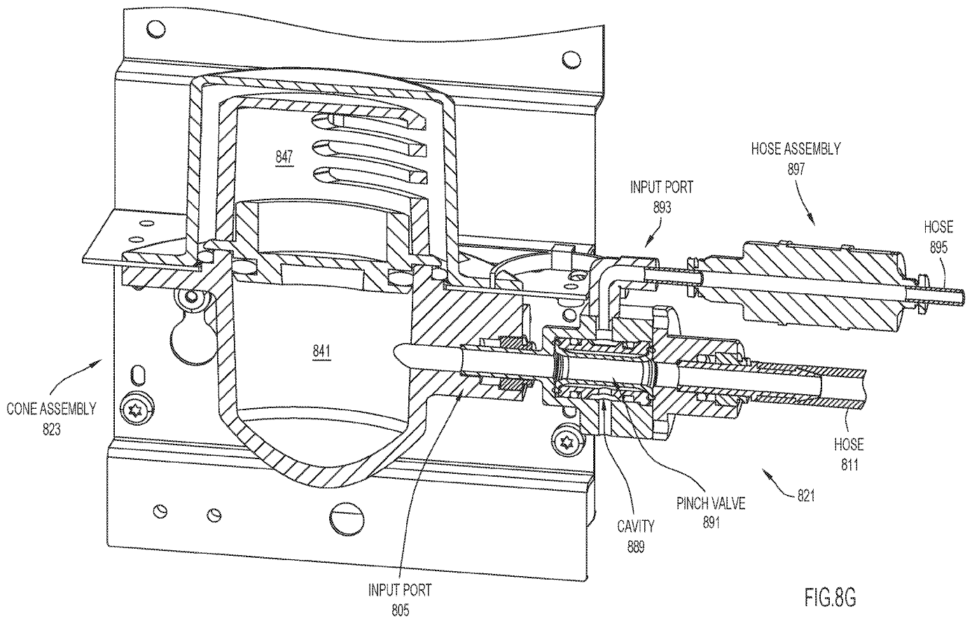

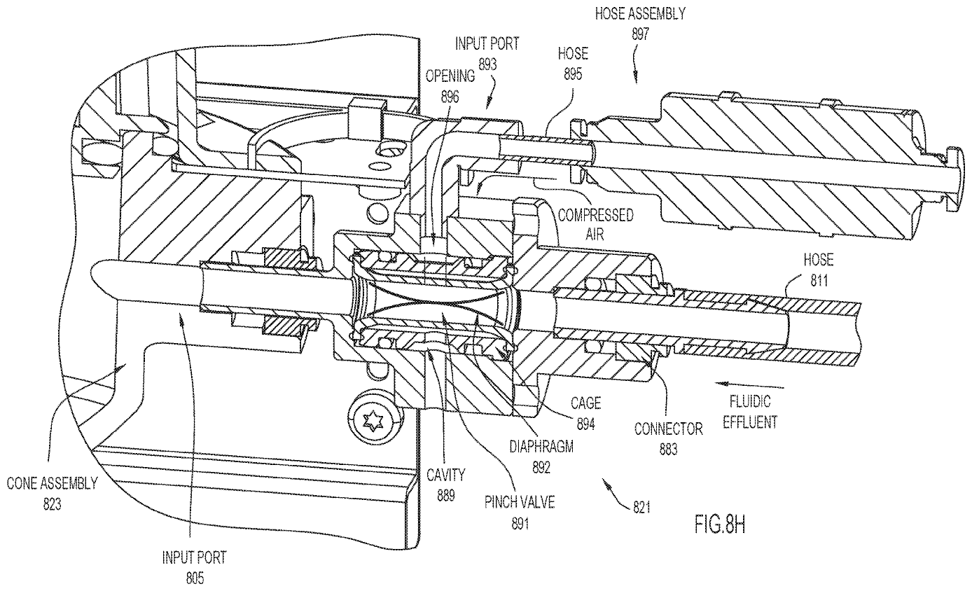

As noted above, the presence of both a centrifugal separation stage and a filtration separation stage, implemented as above, is merely illustrative. The techniques presented herein can be implemented with an effluent management accessory having a refinement stage implemented as a filtration separation stage using a filter (e.g., filter 449, filter 649, etc.) of a filter cartridge assembly (e.g., filter cartridge assembly 425, filter cartridge assembly 625, etc.), a refinement stage implemented using a circuitous pathway (e.g., as described below with reference to FIGS. 8A-8H), or another type of refinement stage. In some other example embodiments, the centrifugal separation stage can operate alone, without any refinement stage.

As noted, FIGS. 8A-8F illustrate one example of an effluent management accessory 801 having a refinement stage implemented as a circuitous pathway. More specifically, FIG. 8A is a view of the effluent management accessory 801 mounted to a sink assembly 890, while FIG. 8B is a view of the effluent management accessory 801 shown separate from the sink assembly 890. FIG. 8C is a cross-sectional view of a junction assembly 871 of the effluent management accessory 801, FIG. 8D is a cross-sectional view of a cone assembly 823 of the effluent management accessory 801, and FIG. 8E is a perspective view of a double cylinder 839 of the cone assembly 823. Finally, FIG. 8F is a diagram illustrating a circuitous pathway 880 of the effluent management accessory 801. For ease of explanation, FIG. 8A-8F will generally be described together below.

In the example of FIGS. 8A-8F, the effluent management accessory 801 is described as generally comprising a drain assembly 817, a cone assembly 823, and a junction assembly 871. Each of these portions will be described in greater detail below. However, it is to be appreciated that the general division of the effluent management accessory 801 into these specific portions is merely for ease of description and that, in alternative arrangements, effluent management accessories presented herein can include different numbers of portions having a number of different structural arrangements.

The effluent management accessory 801 operates by receiving fluidic cleaning effluent (fluidic effluent), which can comprise multi-fluid phase conditions, such as a mixed flow of gas, liquid, and solid, a mixed flow of gas a liquid, gas only, liquid only, etc. As further described below, the effluent management accessory 801 is configured to separate gases (e.g., air) present in the fluidic cleaning effluent from any liquids and solids present in the effluent being discharged by an automatic or manual cleaning process. More specifically, as described further below, a centrifugal separation stage uses a centrifugal separation process to separate liquids and solid portions (particles) of the fluidic effluent from gas portions of the fluidic effluent to produce a gaseous discharge and a non-gaseous discharge. Also as described further below, the centrifugal separation stage is followed by a refinement stage formed by a so-called “circuitous pathway.” The refinement stage in this example, sometimes referred to herein as a “circuitous pathway stage” receives the gaseous discharge (e.g., gas portions and relatively smaller portions of the liquids and solid portions of the fluidic effluent) produced during the centrifugal separation stage, while the relatively non-gaseous discharge (e.g., relatively larger liquids and solid portions of the fluidic effluent are immediately discarded. The circuitous pathway (e.g., via one or more impactor regions, one or more bends, one or more directional changes, forces of momentum and gravity, etc.) operates to separate remaining liquid and/or solid portions (particles) from the separated gas portions, producing a refined gaseous discharge. As such, substantially only the gas portions of the fluidic effluent are safely discharged to the ambient environment/room, while liquid and solid portions of the fluidic effluent are safely discharged into, for example, a sink drain. The structural arrangement of the effluent management accessory 801 is described further below, followed by a more detailed explanation of the functional operation of the effluent management accessory 801.

As noted, the effluent management accessory 801 comprises the cone assembly 823 having at least two input ports, referred to as input port 803 and input port 805, that can each receive effluent. That is, in this example, the input port 803 and the input port 805 are each configured to be fluidically connected to a fluidic cleaning effluent source. In this example, the input port 803 is configured to be fluidically connected to a drain fitting/port (not shown in FIGS. 8A-8F) of a lumen cleaning device (e.g., endoscope reprocessing device's drain port) via a first hose assembly 827, while the input port 805 is configured to be fluidically connected to a distal end (not shown in FIGS. 8A-8F) of one or more lumens (e.g., distal end of an endoscope) via a second hose assembly 829, the junction assembly 871, and a third hose assembly 867.

As noted, the effluent management accessory 801 includes two input ports that can receive effluent. It is to be appreciated that the presence of two input ports is merely illustrative and that other embodiments can include a single input port, or more than two input ports. In one specific example, the input port 803 could be omitted and the drain fitting/port of the lumen cleaning device could be connected to the effluent management accessory 801 so that the fluidic effluent produced thereby enters via input 805 (e.g., the drain fitting/port of the lumen cleaning device could be connected to a junction box upstream from the port 805).

The first hose assembly 827 comprises an end r 809 for connection to the drain port of a lumen cleaning device, a connector (e.g., quick disconnect, not shown in FIG. 8B) for connection to the input port 803, and a hose/tube 807 fluidically connecting the adapter 809 to the connector at the input port 803. The second hose assembly 829 comprises a connector (e.g., quick disconnect, not shown in FIG. 8B) for connection to the input port 805, and a hose/tube 811 fluidically connecting the output port 875 of the junction assembly 871 to the input port 805 of the cone assembly 823. The third hose assembly 867 comprises a distal tip adapter 863 for connection to the distal end of one or more lumens, an adapter 861 for connection to the input port 873 of the junction assembly 871, and a hose 865 fluidically connecting the distal tip adapter 863 to the adapter 861 at the input port 873. In this example, the distal tip adapter 863 (or alternatively, the adapter 861) can include a backflow arrestor (backflow minimizer, not shown), which comprises a cone-shaped piece that minimizes the flow of effluent back into the lumens, as described above.

As shown in FIGS. 8B and 8C, the junction assembly 871 is mounted on a top/upper surface 833 of the sink assembly 890, while the remainder of the effluent management accessory 801 is mounted below/beneath the upper surface 833 of the sink assembly 890. As shown, in this example embodiment, the junction assembly 871 comprises an input port 873 for receiving fluidic effluent via the hose 865 of the third hose assembly 867, an output port 875 for releasing the fluidic effluent via the hose 811 of the second hose assembly 829, an input port 885 for receiving gaseous discharge via a conduit 877 of a conduit assembly 879, and an output port 887. In certain embodiments, the junction assembly 871 also includes a splash shield 851 disposed above the output port 887, which is configured to protect against accidental damage or water splashes, as described elsewhere herein. The splash shield 851 includes openings 853, which form exits to the ambient environment. In general, the openings 853 can be configured to be positioned/facing away from a user and, as described below, gas from the gaseous discharge can pass through the openings 853 and into the ambient environment during operation, while the remaining relatively smaller liquid or solid particles remain inside the housing 845 of the cone assembly 823, the output port 883, the conduit 877, the input port 885, and/or the splash shield 851, as described in further detail below with reference to FIG. 8F. In general, excessive water vapor is removed from the gas to prevent increase of moisture within the ambient environment.

In the specific example of FIGS. 8A-8F, the cone assembly 823 is an element of the centrifugal separation stage and is the interface for the drain port of the lumen cleaning device, the lumen(s) being cleaned, and the sink drain path. As shown, at least part of the cone assembly 823 defines a general conical or tapered internal volume 841 that terminates in a drain port 815. In the example of FIGS. 8A-8F, the cone assembly 823 includes a generally cylindrical upper portion 836, where the input port 803 and the input port 805 are located, and a lower portion 838 with a conical volume. In other embodiments, the upper portion could be omitted such that the entirety of the cone assembly 823 defines a general conical internal volume.

As shown in FIG. 8D, disposed within the internal volume of the cone assembly 823 is a double cylinder 839 having a central aperture 843 (a through-hole), sometimes referred to as a “vortex finder.” In the example of FIGS. 8A-8F, the double cylinder 839 is part of the cone assembly 423, although the double cylinder 839 could be separate from the cone assembly 823 in alternative embodiments. In certain examples, the cone assembly 823 is referred to as having a first end and a second end. As shown, the double cylinder 839 is disposed in the cone assembly 823 adjacent to the first end, while the drain port 815 is disposed at the second end. As such, the cone assembly 823 defines a substantially conical volume between the double cylinder 839 and the drain port 815.

FIG. 8E is an enlarged perspective view of the double cylinder 839, according to an example embodiment. In this example, the double cylinder 839 includes an outer cylinder 840 and an inner cylinder 842, with the central aperture 843 being disposed within the inner cylinder 842. As best seen in FIGS. 8D and 8E, a first end (lower end) of the inner cylinder 842 is offset with respect to a first end (lower end) of the outer cylinder 840. That is, the inner cylinder 842 is shorter in length and the entry point to the inner cylinder 842 is disposed higher in the vertical direction relative to the entry point to the outer cylinder 840, thereby forming an inner chamber 844 between the outer cylinder 840 and the inner cylinder 842.

In addition to the double cylinder 839, the cone assembly 823 comprises a housing 845 (an outer cover) defining an internal volume 847 that is disposed above the central aperture 843 of the double cylinder 839, where the central aperture 843 provides the only fluidic connection between the internal volume 841 and the internal volume 847. That is, the internal volume 847 is disposed in, and surrounded by, the housing 845 (cover). In this example, the top of the housing 845 has a central aperture 881 (a through-hole) disposed above the central aperture 843 of the cone assembly 823, and an output port 883 is disposed in the central aperture 881 of the housing 845. The output port 883 is connected with the input port 885 of the junction assembly 871 via a conduit 877. The conduit assembly 879 comprises the conduit 877, as well as the output port 883 at the cone assembly 823 and the input port 885 at the junction assembly 871.

As shown in FIGS. 8B and 8D, the drain assembly 817 includes a body 857 (or cradle) that is configured to receive and secure the cone assembly 823. The body 857 can swivel to accommodate different device-to-sink orientations. The drain assembly 817 also includes a drain hose/tube 859 having a first end connected to the body 857 and a second end connected to a spigot adapter 819. The drain port 815 of the cone assembly 823 is fluidically connected to a sink drain. That is, the spigot adapter 819 is used to securely connect the drain hose to a sink drain, such as a P-trap. The spigot adapter 819 can be, in certain embodiments, universal for all global standard spigot sizes.

As noted above, the effluent management accessory 801 is configured to perform dynamic separation of gas (e.g., air) from the liquid and solid effluent using centrifugal separation within the cone assembly 823, followed by a circuitous pathway, as described in further detail below with reference to FIG. 8F. In the centrifugal separation stage, the received effluent is impacted along the radial inside wall of the cone assembly 823 and, due to high inertial (centrifugal) forces of liquid, water, and solid particles, the mixture will traverse downward towards the drain port 815, leaving the gases exiting towards the central aperture 843 (vortex finder) of the double cylinder 839 and into the internal volume 847. The double cylinder 839 is an integrated feature in the centrifugal separation stage that provides a boundary between the central aperture 843 and the air radial stream, while not disrupting the internal wall of flow vortices.

Stated differently, in the centrifugal separation stage, the fluidic effluent (received from the endoscope 100 and automated lumen cleaning device 370) is fed tangentially into the cylindrical top of the cone assembly 823 to create rotation. Passing through the cylindrical top where the flow velocity is further accelerated. The centrifugal force separates the stream of non-gaseous discharge of the fluidic effluent while it rotates downward following the surface profile of cone assembly 823. While the stream of gaseous discharge of the fluidic effluent is moved to enter the double cylinder 839 during the rotation.

In summary, the centrifugal separation stage (centrifugal separator) is used to at least partially separate the gas effluent from the solid and liquid effluent, thereby producing what is referred to herein as a “gaseous discharge” (separated and potentially moist gases) and a “non-gaseous discharge” (e.g., liquids and/or solids). The gaseous discharge enters the central aperture 843, while the non-gaseous discharge passes to the drain port 815 (e.g., as a result of gravity) and, eventually, to the drain hose 859 and connected drain. Stated differently, the heavier non-gas particles are separated from the gas via centrifugal force, where the mass of the non-gas particles is larger and thus these larger particles impact the walls of the cone assembly 823 harder to separate them from the gas particles, where gravity pulls the non-gases down into the drain.

In certain examples, the centrifugal separation stage utilizes two conditions that are enabled by a physical arrangement of the effluent management accessory 801. In particular, during operation, the effluent management accessory 801 should be oriented such that the drain port 815 is positioned inferior to the double cylinder 839, thereby allowing gravity to pull the non-gaseous discharge (relatively heavier solids and liquids) down towards the drain port 815, while allowing the relatively lighter gaseous discharge to enter the central aperture 843 of the double cylinder 839. That is, the effluent management accessory 801 is mounted to the sink assembly 890 in an orientation enabling gravity to pull the heavier non-gaseous discharge down towards the drain port 815 and enabling the gaseous discharge to enter the central aperture 843. In addition to the upright orientation, the fluidic resistance of the drain hose 859 should be greater than the fluidic resistance of the circuitous pathway, particularly with a gas only effluent flow (e.g., during air purging). The relatively greater fluidic resistance of the drain hose 859 could be provided by the inclusion of a dynamic shut-off valve (e.g., a hollow ball valve) at the drain port 815, or by providing a minimum water column (dip height) within the drain hose 859, for example.

As noted, the centrifugal separation stage is used to at least partially separate the gas effluent from the solid and liquid effluent. In certain examples, as noted above, the gaseous discharge (e.g., gas effluent and remaining relatively smaller liquid or solid particles) enters the central aperture 843 (vortex finder), and passes into the internal volume 847 within the housing 845 (cover) of the cone assembly 823. However, instead of being filtered via a filter of a filter cartridge assembly (like in the previously described embodiments of FIGS. 4A-4G and FIGS. 6A-6E above), the gaseous discharge is refined/further separated by a refinement stage formed by a so-called “circuitous pathway.” That is, the gaseous discharge can continue to flow through a circuitous pathway in the embodiment of FIGS. 8A-8F, where the circuitous pathway is configured to separate remaining relatively smaller liquid or solid particles of the gaseous discharge from the gas effluent.

In the effluent management accessory 801 described above with reference to FIGS. 8A-8E, the gaseous discharge (gas/air with some liquids/solids suspended therein) flows through a circuitous pathway that comprises one or more impactor regions and/or one or more bends at a location adjacent to a flow direction change (e.g., from an input flow direction to an output flow direction that is orthogonal thereto). For example, a first portion of the gaseous discharge (e.g., larger droplets) flowing in the input flow direction impact at the impactor region and remain at the impactor region, while a second portion of the gaseous discharge (e.g., smaller droplets) remain suspended in the gas and continue to flow in the output flow direction. In general, the circuitous pathway comprises one or more bends in a conduit system, as explained in further detail below with reference to FIG. 8F.

FIG. 8F is a schematic diagram illustrating a circuitous pathway 880 of the effluent management accessory 801, according to an example embodiment. As shown in FIG. 8F (also refer to FIGS. 8B-8D), the circuitous pathway 880 is constrained (and at least partially defined) by a conduit 877 of a conduit assembly 879 that is connected with and extends between the cone assembly 823 (at a first end of the conduit 877) and the junction assembly 871 (at a second end of the conduit 877). As shown in FIG. 8F, the circuitous pathway 880 comprises a first bend 884 of the output port 883 between the cone assembly 823 and the conduit 877, and a second bend 886 of the input port 885 between the junction assembly 871 and the conduit 877. In FIG. 8F, the middle panel shows the overall structure of the effluent management accessory 801 (also refer to FIG. 8B), while the left panel shows an enlarged cross-sectional view of the cone assembly 823 (also refer to FIG. 8D) and the right panel shows an enlarged cross-sectional view of the junction assembly 871 (also refer to FIG. 8C), respectively.

Referring to the left panel in FIG. 8F, when the gaseous discharge flows through the central aperture 843 of the double cylinder 839 of the cone assembly 823, the gaseous discharge enters the internal volume 847 defined by the housing 845. The housing 845 of the cone assembly 823 forms a first impactor region (Region A in FIG. 8F). The gaseous discharge impacts the upper and/or side walls of the housing 845 which, combined with the force of gravity, separates a first portion of liquids/solids from the gas/air. The gaseous discharge (gas/air with the first portion of the liquids/solids removed) then enters the output port 883 in a first input flow direction (substantially vertical).

The output port 883 between the cone assembly 823 and the conduit 877 forms a first bend 884 (Region B in FIG. 8F). When the gaseous discharge enters the output port 883 in the first input flow direction, the gaseous discharge impacts the first bend 884 which, combined with the force of gravity, separates a second portion of liquids/solids from the gas/air. The gaseous discharge (gas/air with the second portion of the liquids/solids removed, but possibly with some smaller portion of the liquids/solids remaining) then exits the output port 883 and enters the conduit 877 in a first output flow direction (substantially horizontal, possibly with a slight upward angle towards the junction assembly 871). In this example, the first output flow direction is substantially orthogonal to the first input flow direction. Thus, the flow of the gaseous discharge changes direction at the output port 883 (first bend 884).

Referring to the middle panel of FIG. 8F, the conduit 877 of the conduit assembly 879 forms a transverse region (Region C in FIG. 8F). As the gaseous discharge flows through the conduit 877 from the output port 883 of the cone assembly 823 to the input port 885 of the junction assembly 871 in the first output flow direction, a third portion of liquids/solids can be separated from the gas/air, due to the flow traversing the length of the conduit 877 combined with the force of gravity. The gaseous discharge (gas/air with the third portion of the liquids/solids removed) then enters the input port 885 in the first output flow direction, which now corresponds to a second input flow direction (still substantially horizontal) at this point.

Referring to the right panel of FIG. 8F, the input port 885 between the conduit 877 and the junction assembly 871 forms a second bend 886 (Region D in FIG. 8F). When the gaseous discharge enters the input port 885 in the second input flow direction, the gaseous discharge impacts the second bend 886 which, combined with the force of gravity, separates a fourth portion of liquids/solids from the gas/air. The gaseous discharge (gas/air with the fourth portion of the liquids/solids removed) then flows in a second output flow direction (substantially vertical) and exits the output port 887 in the junction assembly 871. In this example, the second output flow direction is substantially orthogonal to the second input flow direction. Thus, the flow of the gaseous discharge changes direction at the input port 885 (second bend 886). At this point, the larger particles of liquids/solids above a certain threshold size/mass are removed, and most (if not all) of the smaller particles of liquids/solids below a certain threshold size/mass are also removed.

The splash shield 851 of the junction assembly 871 forms a second impactor region (Region E in FIG. 8F). When the gaseous discharge exits the output port 887 in the second output flow direction, the gaseous discharge impacts the upper and/or side walls of the splash shield 851 which, combined with the force of gravity, separates a fifth portion of liquids/solids from the gas/air. As noted above, most (if not all) of the particles of liquids/solids should already be removed at this point, but any residual particles of liquids/solids that remain suspended in the gas/air can be removed by further collision with the splash shield 851. Finally, the remaining gaseous discharge (gas/air with the fifth portion of the liquids/solids removed) then flows out through the openings 853 in the splash shield 851, thereby exiting the junction assembly 871 and entering the ambient environment. As noted, excessive water vapor is thereby removed from the gas to prevent any dramatic increase of moisture within the ambient environment. The gaseous discharge that exits the effluent management accessory 801 at the junction assembly 871 can also be referred to as a “refined gaseous discharge” in this example embodiment (since a similar separation effect when using a dedicated filter can be achieved by using a circuitous pathway with multiple turns and directional changes instead).

Thus, as the gaseous discharge flows around the turns in the circuitous pathway 880, the particles of greater mass are unable to make the turns and will collide with the opposing walls at the top of the output port 883 (first bend 884) and the side of the input port 885 (second bend 886), thereby being separated/removed from the gas/air. The remainder of the gas/air (and possibly some small particles of lower mass) can flow around the turns without colliding with the walls, and eventually exits the junction assembly 871 via the output port 887 (and through the openings 853 in the splash shield 851). The changes in direction of the circuitous pathway 880 reduce the ability of relatively larger particles of liquids/solids (e.g., above a certain threshold size/mass) to make the turns and pass through, although some relatively smaller particles of liquids/solids (e.g., below a certain threshold size/mass) may be able to make the turns and pass through. When the multiple elements of the circuitous pathway 800 are added together (i.e., the impact at the housing 845, the impact and directional change at the first bend 884 of the output port 883, the extending length of the conduit 877, the impact and directional change at the second bend 886 of the input port 885, the impact at the splash shield 851), the individual elements in combination can effectively provide a filtering mechanism with a defined cut-off size/mass with respect to any liquids/solids that are suspended within gas/air of a gaseous discharge.

It should be appreciated that a “strict” orthogonal relationship between the first input flow direction and the first output flow direction, and between the second input flow direction and the second output flow direction, is not necessarily required. In one non-limiting example embodiment, the first bend 884 at the output port 883 and the second bend 886 at the input port 885 can be substantially perpendicular (e.g., 90 degree angle). However, a greater or lesser angle than 90 degrees can be used in some other example embodiments. For example, an obtuse angle greater than 90 degrees can be used, particularly when the conduit 877 is not strictly horizontal but rather slopes upward slightly, in the direction from the output port 883 at the cone assembly towards the input port 885 at the junction assembly 871 (e.g., from left to right in the middle panel of FIG. 8F). In some examples, the angle of the first bend 884 at the output port 883 may or may not be identical to the angle of the second bend 886 at the input port 885.

By including a circuitous pathway with one or more changes in flow direction in an effluent management accessory, such as the circuitous pathway 880 of FIG. 8F (also refer to FIGS. 8B-8D) that is at least partially defined by the first bend 884 of the output port 883 at the cone assembly 223, the conduit 877 of the conduit assembly 879, and the second bend 886 of the input port 885 at the junction assembly 871, any liquids and/or solids that are mixed in with gas/air can effectively be “separated” out (i.e., filtered, separated, removed, restricted, reduced, etc.) from a gaseous discharge.

In summary, the circuitous pathway stage using the circuitous pathway 880 (e.g., with the Regions A-E, first bend 884 at output port 883, second bend 886 at input port 885, etc. as shown in FIG. 8F) manages the solid and liquid particles that were not collected by the centrifugal separation stage using the cone assembly 823 (e.g., via the double cylinder 839 as shown in FIGS. 8D-8F). In general, these particles have a size that is below the “cut-off” diameter of the centrifugal separation stage Collection Efficiency Threshold (CET). As noted above, a filter cartridge assembly (filter) is not provided in the specific example of FIG. 8A-8F, and the centrifugal separation stage instead operates in conjunction with the circuitous pathway stage to manage the solid and liquid particles from the effluent. As noted, the circuitous pathway stage can be considered optional and may or may not be provided, and the centrifugal separation stage can operate alone to manage the solid and liquid particles from the effluent in some example embodiments.

Referring to FIG. 8B and as further described below with reference to FIGS. 8G and 8H, in certain embodiments, the hose assembly 829 (between the junction assembly 871 and the cone assembly 823) includes a connector or adapter 821 with a pinch valve 891. The connector 821 includes a third input port, referred to as input port 893, that is configured to receive gas (compressed air) from a gas source (e.g., compressed air source, not shown in FIG. 8B and FIGS. 8G-8H) via a hose/tube 895 of a fourth hose assembly 897. In this example, the gas/air passes through the input port 893 and enters the connector 821 to actuate the pinch valve 891. As noted below, the gas/air source and the pinch valve 891 can be used to test whether one or more connections of the effluent management accessory 801 provide a proper seal, as described below with reference to FIGS. 8G and 8H.

More specifically, FIG. 8G is a schematic diagram showing a cross-sectional view of the pinch valve 891 disposed in the connector 821, according to another example embodiment. FIG. 8H is an enlarged cross-sectional view showing components of the pinch valve 891 of FIG. 8G in greater detail. As shown in FIG. 8G, the pinch valve 891 is mounted within a cavity 889 (recess) of the connector 821 and is fluidically coupled to the hose 811 at a first end thereof and the internal volume 841 of the cone assembly 823 at a second end thereof located at the input port 805. The pinch valve 891 is configured to control the flow of fluidic effluent from the hose 811 through the input port 805 and into the cone assembly 823. The pinch valve 891 can define an opening through which the fluidic effluent can flow, and the opening defined by the pinch valve 891 can be closed stop the flow of fluidic effluent. For example, a component of the pinch valve 891 can flex or deform to stop the flow of fluidic effluent through the input port 805. In this example, the pinch valve 891 is configured to transition between an open configuration to enable fluidic effluent flow through the input port 805 and a closed configuration to block fluidic effluent flow through the input port 805 and preventing the fluidic effluent from entering the cone assembly 823.

As best seen in FIG. 8H, the pinch valve 891 comprises a diaphragm 892 (elongate tubular diaphragm) and a cage 894 (elongate sleeve) in which the diaphragm 892 can be inserted. The cage 894 is configured to be disposed around the diaphragm 892 in a sealing engagement with the diaphragm 892 at an inner surface of the cage 894 that is configured to accommodate a profile of the diaphragm 892, thereby securing the diaphragm 892 within the cage 894. The cage 894 includes an opening 896 (a through-hole) configured to fluidically couple the diaphragm 892 to the input port 893 that is adjacent to an outer surface of the cage 894. The opening 896 enables flow of a working fluid (e.g., a gas, compressed air, etc.) to abut against and impart a force onto the diaphragm 892 to transition the diaphragm 892 of the pinch valve 891 to the closed configuration. The diaphragm 892 and the cage 894 sealingly engage one another to block undesirable flow of working fluid out of the pinch valve 891, thereby forcing the working fluid to flow against the diaphragm 892 to transition the diaphragm 892 to the closed configuration.

The diaphragm 892 can have a body with a tubular profile that defines an opening, and can be composed of a flexible or pliable material (e.g., an elastomer, medical grade silicone to provide corrosion resistance, etc.), such that the walls of the diaphragm 892 can be compressed toward one another to reduce a size of the opening defined by the diaphragm 892, thereby restricting or blocking fluid flow through the diaphragm 892. The walls of the diaphragm 892 can also be moved away from one another to increase the size of the opening defined by the diaphragm 892, thereby allowing fluid flow through the diaphragm 892. The material of the diaphragm 892 can also be sufficiently resilient such that absent a force imparted onto the diaphragm 892, the diaphragm 892 can adjust toward a base shape or profile.

As discussed herein, the diaphragm 892 can flex or deform to open or close the opening, and the sufficiently pliable material of the diaphragm 892 can enable some flexure via a working fluid (e.g., compressed air). A gas/air source (not shown) can be fluidically connected to the input port 893 via the hose 895 of the fourth hose assembly 897, and the input port 893 is fluidically connected with the opening 896 (through-hole) in the cage 894. The opening 896 can expose a portion of the diaphragm 892 disposed within the cage 894, thereby fluidically coupling (e.g., pneumatically coupling) the diaphragm 892 to the input port 893. The gas/air source is configured to direct working fluid (compressed air) towards the pinch valve 891, through the hose 895 and the input port 893, then through the opening 896 defined in the cage 894 and against the diaphragm 892. The working fluid (compressed air) output against the diaphragm 892 can provide a sufficient force to cause the diaphragm 892 to flex and adjust the opening, and compress the diaphragm 892 from an open configuration to a closed configuration (shown in phantom lines in FIG. 8H), which can slow the flow of fluidic effluent through the opening defined by the diaphragm 892. Absent the working fluid (compressed air) output by the gas/air source, the diaphragm 892 can expand and transition out of the closed configuration and return to the open configuration to increase the rate of fluidic effluent through the opening defined by the diaphragm 892. As such, the gas/air source can be operated (e.g., manually by a user, automatically via a controller) to adjust the flow of fluidic effluent through the pinch valve 891, and hence, through the input port 805 into the cone assembly 823.

The cage 894 can also help secure the pinch valve 891 within the cavity 889 of the input port 805, and can be composed of a rigid material (e.g., metal, copolymer, hard plastic, acetal, etc.) that avoids substantial deformation and provides desirable machining characteristics for forming the shape of the cage 894, the opening 896, and so forth. Additionally, the rigid construction of the cage 894 can block deformation of the cage 894 while working fluid (e.g., a gas, compressed air, etc.) flows through the opening 896, such that the profile of the cage 894 can be maintained when the working fluid flows through the opening 896.

In operation, the pinch valve 891 described above with reference to FIGS. 8G-8H can be used to check whether the various connections of the effluent management accessory 801 are made properly. For example, the pinch valve assembly 891 could be activated to close the diaphragm 892. If the various connections of the effluent management accessory 801 upstream from the pinch valve assembly 891 are correctly made (e.g., the connections between the endoscope 100 and the connector 821, including distal tip adapter 863, adapter 861, etc.), there should be an increase in pressure that can be detected at the attached endoscope or lumen cleaning device when compressed air is subsequently applied to the endoscope lumens for leak testing purposes. However, little to no increase in pressure (when compressed air is subsequently applied to the endoscope lumens for leak testing purposes), can indicate that one or more of the various upstream connections is/are not made properly. In such circumstances, an alert (e.g., audible, visible, tactile, etc.) can be provided to recheck one or more of the various connections of the effluent management accessory 801 (e.g., for the endoscope 100 and so forth). Thus, the pinch valve 891 and the corresponding techniques described above can be used as an initial connection check (i.e., a preliminary verification step), prior to commencing normal operation of the effluent management accessory 801, for example.

One notable aspect of the effluent management accessory 801 is the junction assembly 871. As explained above, the junction assembly 871 serves several purposes, including operating as the connection or interface point (junction) between the effluent source and the cone assembly (e.g., where the separation is performed). In addition, the junction assembly 871 is also the exit point for the effluent gas, after passing through the centrifugal separation stage and the circuitous pathway stage. As noted, the openings in the junction assembly 871 are also oriented so that the refined gaseous discharge produced by the effluent management accessory 801 are discharged/released away from a user (e.g., towards the wall of the room). Significantly, the junction assembly 871 is the only portion of the effluent management accessory 801 that would be visible during use (other of the connections the effluent source) because it mounted on the top/upper surface 833 of the sink assembly 890, while the remainder of the effluent management accessory 801 is mounted below/beneath the upper surface 833 of the sink assembly 890. In this form, the junction assembly 871 provides a “clean” install of the effluent management accessory 801 where most of the accessory is hidden from view, yet still provides a convenient way to connect an effluent surface and release the gaseous effluent after separation.

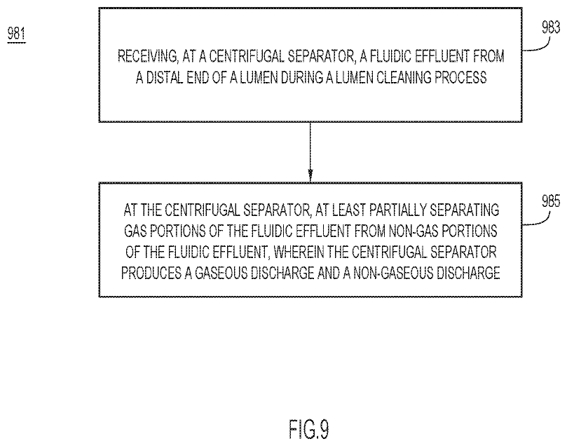

FIG. 9 is a flowchart of an example method 981, in accordance with certain embodiments presented herein. Method 981 begins at 983 where a centrifugal separator of an effluent management accessory (e.g., effluent management accessory 301, 401, 601, 801, etc.) receives a fluidic effluent from a distal end of a lumen during a lumen cleaning process. At 985, the centrifugal separator at least partially separates gas portions of the fluidic effluent from non-gas portions of the fluidic effluent to produce a gaseous discharge and a non-gaseous discharge.

Certain aspects of the techniques presented herein have been described with reference to various descriptions of fluid dynamics. It is to be appreciated that these various descriptions are provided for purposes of illustration and that the innovation presented herein works regardless of the believed understanding of the fluid dynamics.

As should be appreciated, while particular uses of the technology have been illustrated and discussed above, the disclosed technology can be used with a variety of devices in accordance with many examples of the technology. The above discussion is not meant to suggest that the disclosed technology is only suitable for implementation within systems akin to that illustrated in the figures. In general, additional configurations can be used to practice the processes and systems herein and/or some aspects described can be excluded without departing from the processes and systems disclosed herein.

This disclosure described some aspects of the present technology with reference to the accompanying drawings, in which only some of the possible aspects were shown. Other aspects can, however, be embodied in many different forms and should not be construed as limited to the aspects set forth herein. Rather, these aspects were provided so that this disclosure was thorough and complete and fully conveyed the scope of the possible aspects to those skilled in the art.

As should be appreciated, the various aspects (e.g., portions, components, etc.) described with respect to the figures herein are not intended to limit the systems and processes to the particular aspects described. Accordingly, additional configurations can be used to practice the methods and systems herein and/or some aspects described can be excluded without departing from the methods and systems disclosed herein.

According to certain aspects, systems and non-transitory computer readable storage media are provided. The systems are configured with hardware configured to execute operations analogous to the methods of the present disclosure. The one or more non-transitory computer readable storage media comprise instructions that, when executed by one or more processors, cause the one or more processors to execute operations analogous to the methods of the present disclosure.

Similarly, where steps of a process are disclosed, those steps are described for purposes of illustrating the present methods and systems and are not intended to limit the disclosure to a particular sequence of steps. For example, the steps can be performed in differing order, two or more steps can be performed concurrently, additional steps can be performed, and disclosed steps can be excluded without departing from the present disclosure. Further, the disclosed processes can be repeated.

Although specific aspects were described herein, the scope of the technology is not limited to those specific aspects. One skilled in the art will recognize other aspects or improvements that are within the scope of the present technology. Therefore, the specific structure, acts, or media are disclosed only as illustrative aspects. The scope of the technology is defined by the following claims and any equivalents therein.

It is also to be appreciated that the embodiments presented herein are not mutually exclusive and that the various embodiments may be combined with another in any of a number of different manners.

1-26. (canceled)

27. A method, comprising:
receiving, at a centrifugal separator, a fluidic effluent from a distal end of a lumen during a lumen cleaning process; and
at the centrifugal separator, at least partially separating gas portions of the fluidic effluent from non-gas portions of the fluidic effluent to produce a gaseous discharge and a non-gaseous discharge.

28. The method of claim 27, further comprising:
separating gas portions of the gaseous discharge from non-gas portions of the gaseous discharge.

29. The method of claim 28, wherein separating gas portions of the gaseous discharge from non-gas portions of the gaseous discharge comprises:
providing the gaseous discharge to a filter cartridge assembly fluidically connected to the centrifugal separator; and
at the filter cartridge assembly, separating the gas portions of the gaseous discharge from the non-gas portions of the gaseous discharge.

30. The method of claim 28, wherein separating gas portions of the gaseous discharge from non-gas portions of the gaseous discharge comprises:
providing the gaseous discharge to a conduit of a circuitous pathway fluidically connecting the centrifugal separator to a junction assembly; and
separating the gas portions of the gaseous discharge from the non-gas portions of the gaseous discharge via the circuitous pathway as the gaseous discharge flows from the centrifugal separator to the junction assembly.

31. The method of claim 27, further comprising:
providing the non-gaseous discharge to a drain assembly fluidically connected to the centrifugal separator.

32. The method of claim 27, wherein the centrifugal separator includes at least one input port, and wherein the method further comprising:
fluidically connecting the at least one input port to the distal end of the lumen.

33. The method of claim 32, wherein fluidically connecting the at least one input port to a distal end of the lumen comprises:
fluidically connecting the at least one input port to a distal end of at least one internal lumen of an endoscope.

34. The method of claim 32, wherein the centrifugal separator includes a plurality of input ports, and wherein the method further comprises:
fluidically connecting at least one input port of the plurality of input ports to a lumen cleaning device.

35. The method of claim 27, wherein a pinch valve assembly is disposed between the centrifugal separator and the distal end of the lumen, and wherein the method comprises:
closing the pinch valve assembly; and
monitoring a pressure between the pinch valve assembly and a device coupled to a proximal end of the lumen.

36. An apparatus, comprising:
a centrifugal separator comprising at least one input port configured to be connected to at a distal end of a lumen and to receive a fluidic effluent produced during cleaning of the lumen,
wherein the centrifugal separator is configured to at least partially separate gas portions of the fluidic effluent from non-gas portions of the fluidic effluent, wherein the centrifugal separator produces a gaseous discharge and a non-gaseous discharge.

37. The apparatus of claim 36, wherein the centrifugal separator comprises:
a double cylinder disposed within a housing adjacent to a first end of the housing; and
a drain port located at a second end of the centrifugal separator, wherein a filter cartridge assembly is attached to a first end of the centrifugal separator.

38. The apparatus of claim 37, wherein the filter cartridge assembly includes an internal volume, one or more openings to an ambient environment, and a filter disposed between the internal volume and the one or more openings.

39. The apparatus of claim 38, wherein the filter is a coalescing filter.

40. The apparatus of claim 38, wherein the housing defines a substantially conical volume between the double cylinder and the drain port, and wherein the double cylinder includes a central aperture fluidically connecting the substantially conical volume to the internal volume of the filter cartridge assembly.

41. The apparatus of claim 36, wherein the centrifugal separator produces a gaseous discharge and a non-gaseous discharge, and wherein the apparatus further comprises:
a second separator configured to further separate gas portions of the gaseous discharge from non-gas portions of the gaseous discharge.

42. The apparatus of claim 41, wherein the second separator comprises:
a circuitous pathway configured to separate gas portions of the gaseous discharge from non-gas portions of the gaseous discharge, wherein the circuitous pathway includes at least:
a conduit fluidically connected to the centrifugal separator, and
one or more ports disposed at one or more ends of the conduit and forming one or more bends.

43. The apparatus of claim 36, further comprising a backflow arrestor connected between the at least one input port and the distal end of the lumen.

44. The apparatus of claim 43, further comprising:
a hose assembly having a first end connectable the distal end of the lumen, a second end connectable to the at least one input port, and a hose connecting the first end to the second end,
wherein the backflow arrestor is disposed at the first end of the hose assembly.

45. The apparatus of claim 37, further comprising a dynamic shut-off valve disposed proximate to the drain port, wherein the dynamic shut-off valve is configured to minimize a flow of gas into the drain port.

46. The apparatus of claim 36, further comprising:
a pinch valve assembly disposed between the at least one input port and the distal end of the lumen.
